(19) United States
(12) Patent Application Publication
(10) Pub. No.: US 2013/0281409 A1
(43) Pub. Date: Oct. 24, 2013

(54) Myelin Sheath Fatty Acids that Resolve Neuroinflammation

(71) Applicants: The Board of Trustees of the Leland Stanford Junior University, (US); Department of Veterans Affairs, (US)

(72) Inventors: Lawrence Steinman, Stanford, CA (US); Peggy Pui-Kay Ho, Cupertino, CA (US); William H. Robinson, Palo Alto, CA (US); Jennifer L. Kanter, Boston, MA (US)

(21) Appl. No.: 13/847,364

(22) Filed: Mar. 19, 2013

Related U.S. Application Data
(60) Provisional application No. 61/612,805, filed on Mar. 19, 2012.

(51) Int. Cl. A61K 31/661; A61K 31/20

(52) U.S. Cl. CPC A61K 31/661; A61K 31/20
USPC 514/114; 514/121; 514/558

(57) ABSTRACT

Methods are provided for decreasing inflammatory disease in a subject by administering an effective dose of a lipid, fatty acid, or analog thereof.

### GOVERNMENT RIGHTS

This invention was made with Government support under NS055997 awarded by the National Institutes of Health. The Government has certain rights in this invention.

### INTRODUCTION

Lipids are important targets of immune responses in a variety of inflammatory and autoimmune diseases. However, immune responses to lipids have been studied much less extensively than responses to proteins largely due to lack of enabling technologies. Existing methods to study immune responses against lipids are hindered by the large number of potential lipid antigens, the hydrophobicity of lipids, and the technical difficulty of detecting B and T cell responses directed against lipids.

In multiple sclerosis (MS) aberrant adaptive immune responses target and destroy the myelin sheath. Although MS is classically considered a T-cell-driven disease, autoantibodies are increasingly recognized as contributing to its pathogenesis. Several studies on MS demonstrate T-cell and antibody reactivity to lipids, which comprise over 70% of the myelin sheath. Synthesis of anti-lipid antibodies within the central nervous system (CNS) is associated with an aggressive disease course in MS, and, in an experimental model of MS, anti-lipid antibodies both induced demyelination and prevented remyelination. Despite recent interest in the potential pathogenicity of antibodies directed against brain lipids, the specificities of the anti-lipid antibody responses in MS remain undefined.

### Publications

Autoimmune responses directed against phospholipids and gangliosides contribute to the pathogenesis in systemic lupus erythematosus and Guillain Barré syndrome, respectively (Fredman (1998) Ann N Y Acad Sci 845, 341-52). Despite reports of anti-myelin lipid responses in MS, the role of anti-lipid autoimmunity in MS remains controversial (Giovannoni et al. (2000) Ann Neurol 47, 684-5). Most lipids are presented to T cells bound to CD1 molecules (Moody et al. (2005) Nat Rev Immunol 5, 387-99) and CD1 expression is increased in CNS lesions in both MS and EAE (Battistini et al. (1996) J Neuroimmunol 67, 145-51 (1996).

### SUMMARY OF THE INVENTION

The present invention is drawn to methods for decreasing inflammatory disease in a subject by administering an effective dose of a lipid, fatty acid, or analog thereof.

In some embodiments of the invention, the therapeutic agent is a lipid having the structure:

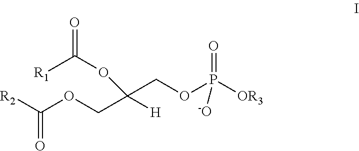

where R₁ and R₂ are independently selected from a linear or branched C₃-C₁₀₀ alkyl; preferably a C₁-C₃₀ alkyl optionally substituted with halo, hydroxy, alkoxy, amino, alkylamino, dialkylamino, sulfate, or phosphate, and which may by saturated, or mono- or di-unsaturated, e.g. 18:0, 24:0 and 24:1. In some embodiments R₁ or R₂ is (Z)-octadec-9-ene. In some embodiments R₂ is hexadecane.

R₃ is selected from H, —CH₂CH₂NH₃ (ethan-1-amine), and serine (2-aminobutanoic acid).

R₄ is absent, or when R₃ is H, R₄ may be

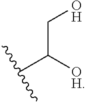 [SMILES: *C(O)CO]

In other embodiments, the therapeutic agent is a fatty acid, e.g., including without limitation palmitic acid, ethyl palmitate, sebacic acid, octanoic acid, methyl octanoate, and suberic acid.

An oxidized lipid of formula I or fatty acid is administered in a therapeutic dose to an individual to inhibit or decrease the adverse effects of an inflammatory disease, for example, a demyelinating disease. In some embodiments the therapeutic agent is delivered every 2 days, e. about every 48 hours. Administration may be systemic, e.g. i.v., or localized, e.g. intracranially, e.g. by CED, etc.

It is shown that administration of a therapeutic dose of these specific lipid molecules, which are optionally administered in conjunction with a tolerizing adjuvant, can prevent or decrease autoimmune responses, e.g. in the treatment or prevention of demyelinating autoimmune diseases, etc.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Autoantibody targeting of lipids is higher in MS CSF than in normal CFS, and the autoantibody-targeted lipid PGPC attenuates EAE. (A) Lipid-array profiling of IgG+IgM antibody reactivity in CSF samples from MS patients (RRMS, relapsing remitting MS; SPMS, secondary progressive MS; PPMS, primary progressive MS), healthy controls (HC), and other neurological disease (OND) controls. Lipid hits with the lowest FDR (q=0.048) were clustered according to their reactivity profiles. Sample type and ID number are shown above the heatmap, and the lipids targeted are shown to the right of the heatmap. (B) Clinical EAE scores of mice coinjected subcutaneoulsy with PLP₁₃₉₋₁₅₁ and 6 μg/injection of PGPC (day 0); on days 4 and 7 after immunization, PGPC was injected intraperitoneally. Arrows indicate when PGPC was administered. Each point represents the mean±SEM, and results are representative of 4 independent experiments. *P<0.05 Mann-Whitney test, vehicle control (n=5) vs PGPC (n=5).

FIG. 2. PGPC-related lipids with non-bulky polar head groups are targeted by antibodies in CSF of MS patients. (A) Mini-Array I: IgG antibody reactivity to various glycero-3-phosphocholine lipids in CSF samples from patients with relapsing remitting MS (RRMS) and from control patients with other neurological disease (OND). Lipid hits with the lowest FDR (q=0.029) were clustered according to their reactivity profiles. Sample type and ID number are shown above the heatmap, and the lipids targeted are shown to the right of the heatmap. (B) Structures of lipid hits in (A). (C) Mini-Array II: IgG antibody reactivity to lipids constituting polar head-group and side-chain modifications of PGPC in CSF samples from RRMS patients and OND controls. Lipid hits with the lowest FDR (q=0.016) were clustered according to their reactivity profiles. All of the lipids screened in Mini Array I and II are listed in Table 1. (D) Left column, structures of the lipid targets identified in (C), with green boxes around the polar head group; right column, structures of the lipids that were not targeted, with red boxes around the polar head group.

FIG. 3. Levels of POPS, PGPC, azPC, and azPC ester are higher in MS brain than in healthy brain. (A) Negative-ion electrospray ionization mass spectrometric analyses of palmitoyl oleoyl phosphatidylserine (POPS) in lipid extracts of normal-appearing white matter from an age-matched healthy control brain (left panel) and of an active lesion from a brain afflicted with relapsing remitting MS (right panel). DMPS, dimyristoyl phosphatidylserine; IS, internal standard. (B) Single-reaction monitoring analysis of PGPC azPC ester, azPC, and POPS levels in MS samples and age-matched healthy controls. Controls: 6 age-matched individuals with no signs of neurological disease; MS: 3 patients with relapsing remitting MS; 1 patient with secondary progressive MS; and 2 patients with chronic MS. *P<0.05 by unpaired Student's t-test.

FIG. 4. Administration of lipids that are targeted by autoantibodies and whose levels are decreased in MS attenuate ongoing EAE and T-cell activation. (A) Clinical scores of PLP₁₃₉₋₁₅₁-immunized SJL mice treated at the peak of EAE with 100 μg/injection of POPS (n=10), PGPC (n=10), azPC ester (n=10), azPC (n=10), or vehicle alone (n=10). Arrows indicate injections of lipid or vehicle. Each point represents the mean clinical score ±SEM (* denotes time points at which P<0.05 by Mann-Whitney test comparing vehicle treatment vs. lipid treatment). (B) Cytokine production by and (C) proliferation of naive MBP_{Ac1-11}-TCR transgenic splenocytes stimulated with 2 μg/ml MBP_{Ac1-11} in the presence of 30 μg/ml of lipid (structures shown in D), as indicated. Values are the mean±SEM of triplicates. Results are representative of 3 independent experiments. (#P<0.01, *P<0.05 by Student's t-test, each lipid plus MBP_{Ac1-11} vs. MBP_{Ac1-11} alone).

FIG. 5. POPS, PGPC, azPC, and azPC ester induce apoptotic signaling pathways and T-cell apoptosis. (A) Annexin V and 7AAD staining of CD3⁺ T cells purified from wild-type B6 mice and stimulated with plate-bound anti-CD3 and anti-CD28 antibodies for 48 h with or without 30 μg/ml of lipid. Cells are gated on CD4⁺ T cells, and results are representative of 3 experiments. (B) Immunoblot analysis of phopsho-ERK1/2, phospho-IKKα/β, phospho-p65, IκBa, phospho-Bcl-2, phospho-Bad, and phospho-Bim in lysates of wild-type CD3⁺ T cells stimulated with plate-bound anti-CD3 and anti-CD28 antibodies for 15 min (left panel) and 24 h (right panel) in the presence of 30 μg/ml of lipid, as indicated. Blots are representative of two independent experiments. (C) TUNEL staining of brain and spinal-cord tissue from mice immunized with PLP₁₃₉₋₁₅₁ peptide (to induce EAE) and treated for 12 h with azPC, POPS, or vehicle on day 15 after immunization. Arrows indicate TUNEL-positive (bright pink/red) infiltrating cells in the perivascular cuffs of lesions from mice with active EAE. Original magnification, ×400. (D) Quantification of TUNEL-positive infiltrating cells in brain and spinal cord shown in panel (C).

FIG. 6. Palmitic acid, a non-polar side chain of 1-Palmitoyl phospholipids, suppresses T-cell proliferation and inflammatory cytokine production, induces T-cell apoptosis, and attenuates EAE. (A) Structure of palmitic acid. (B) Proliferation and (C) cytokine production of naive T cells purified from wild-type B6 mice and stimulated for 48 h with 5 μg/ml of anti-CD3 and anti-CD28 antibody and either 30 μg/ml of lipid or 0.25 mM of palmitic acid. Values are the mean±s.e.m. of triplicates. Results are representative of 3 independent experiments (*P<0.05 by Student's t-test, compared to anti-CD3/anti-CD28 alone). (D) Apoptosis (indicated by annexin V and 7AAD staining) of CD3⁺ T cells purified from wild-type B6 mice and stimulated with plate-bound anti-CD3 and anti-CD28 antibodies for 48 h alone or with ethanol (ETON) or 0.25 mM of palmitic acid (PA). Cells are gated on CD4⁺ T cells. (E) Clinical scores of PLP₁₃₉₋₁₅₁-immunized SJL mice treated at the peak of EAE with 100 μg/injection of palmitic acid (n=10), or vehicle alone (n=10). Arrows indicate injections of palmitic acid or vehicle. Each point represents the mean clinical score (*P<0.05 by Mann-Whitney test comparing vehicle treatment vs. palmitic acid treatment).

FIG. 7. Cerebrospinal fluid levels of IgG and IgM are elevated in MS. Levels of total IgG (A) or total IgM (B) in cerebrospinal fluid (CSF) from patients with relapsing remitting MS (RRMS) or other (non-inflammatory) neurological disease (OND). *P 0.05 by Student's t test. (C) Comparison of levels of total IgG in CSF from patients with RRMS, OND, or secondary progressive MS (SPMS), or from healthy controls (HC). *P>0.05 by one-way ANOVA.

FIG. 8. PGPC, but not sphingomyelin, ameliorates established EAE. (A) EAE severity in PLP139-151-immunized mice administered PGPC or sphingomyelin during the immunization. Six micrograms of PGPC or sphingomyelin were administered during the immunization of mice with PLP139-151, and on days 4 and 7 after the immunization. During the immunization, lipids were mixed with the PLP139-151-CFA emulsion and injected subcutaneously. On days 4 and 7, lipids were solubilized in 0.05% Tween-20 in PBS and injected into the intraperitoneal cavity. (B) EAE severity in PLP139-151-immunized mice administered PGPC, sphinogmyelin, or vehicle at the onset of disease. Upon developing clinical signs of EAE, mice were intravenously administered 100 μg of PGPC (n=9) or sphingomyelin (n=9), or vehicle alone (n=10) for a total of five intravenous injections. Each point represents the mean±s.e.m.+P<0.05 by Mann-Whitney test comparing vehicle-treated (n=5) and PGPC-treated (n=5) mice; □P<0.05 by Mann-Whitney test comparing vehicle-treated (n=5) and sphingomyelin-treated (n=5) mice.

FIG. 9. PGPC treatment of mice with EAE suppresses T-cell activation. (A) Expression of CD69 (an early marker of activation) on CD4+ and CD8+ propidium iodide-negative (i.e. live) lymphocytes isolated from PGPC—or vehicle-treated EAE mice and cultured for 4 days with 10 μg/ml of PLP139-151. The ratio of CD69+CD4+:CD69−CD4+ is 14.2%:39.6% for lymph node cells from vehicle-treated EAE mice, and 7.96%:55.2% for cells from PGPC-treated EAE mice. CD69+CD4+ cells and CD69+CD8+ cells are boxed in red, and percentages of cells in each quadrant are displayed. (B) Proliferation and (C) cytokine production of splenocytes isolated from the vehicle- or PGPC-treated EAE mice in panel A. Splenocytes from PGPC-treated mice (gray bars) secreted lower levels of IFN-γ and TNF in response to PLP139-151. *P<0.05, respectively, by Student's t-test comparing PLP139-151-stimulated cells from PGPC-treated mice with PLP139-151-stimulated cells from vehicle-treated mice.

FIG. 10. DGPC binds to the PVDF membrane used in the lipid arrays. Luxol fast blue staining of 1-Palmitoyl-2-Azelaoyl-sn-Glycero-3-Phosphocholine (azPC), 1,2-Dipropionoyl-sn-Glycero-3-Phosphocholine (DGPC), L-□-phosphatidylserine (PS), and Cardiolipin (CL) attached to the PVDF membrane used in the lipid antigen arrays in FIGS. 1A and 2A, C.

FIG. 11. Levels of antibodies against PGPC, POPS, azPC-ester, and azPC are higher in RRMS than in OND cerebrospinal fluid. ELISA analysis of autoantibodies to (A) PGPC; (B) POPS; (C) azPC ester; and (D) azPC in cerebrospinal fluid from patients with relapsing remitting MS (RRMS) or other neurological disease (OND). Statistical analysis was performed using the unpaired two-tailed Student's t-test, *P<0.05.

FIG. 12. PGPC is present in human brain. LC-HRMS detection of PGPC in human brain sample obtained at autopsy. (A) Accurate mass agreement with calculated mass, (B) retention time on the column in comparison to authentic standards, and (C) MS/MS fragmentation patterns in comparison to authentic standards.

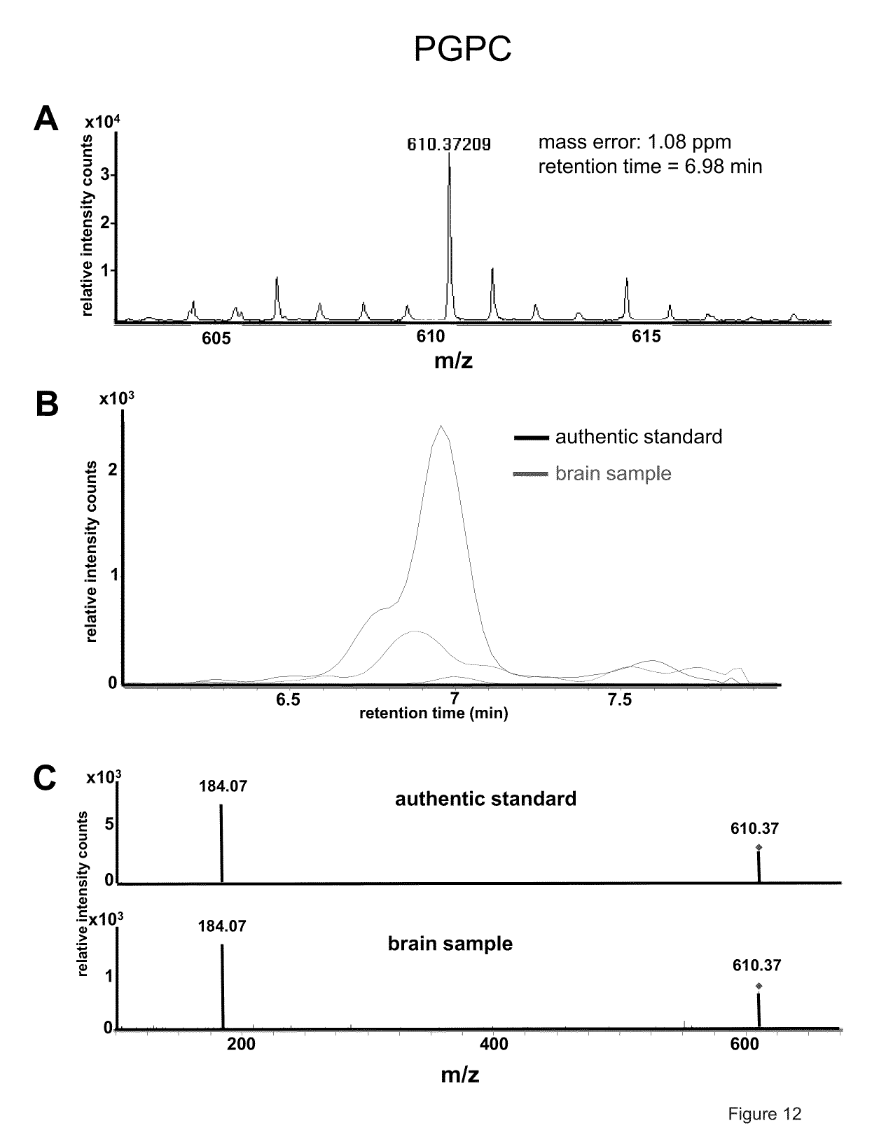

FIG. 13. azPC is present in human brain. LC-HRMS detection of azPC in human brain sample obtained at autopsy. (A) Accurate mass agreement with calculated mass, (B) retention time on the column in comparison to authentic standards, and (C) MS/MS fragmentation patterns in comparison to authentic standards.

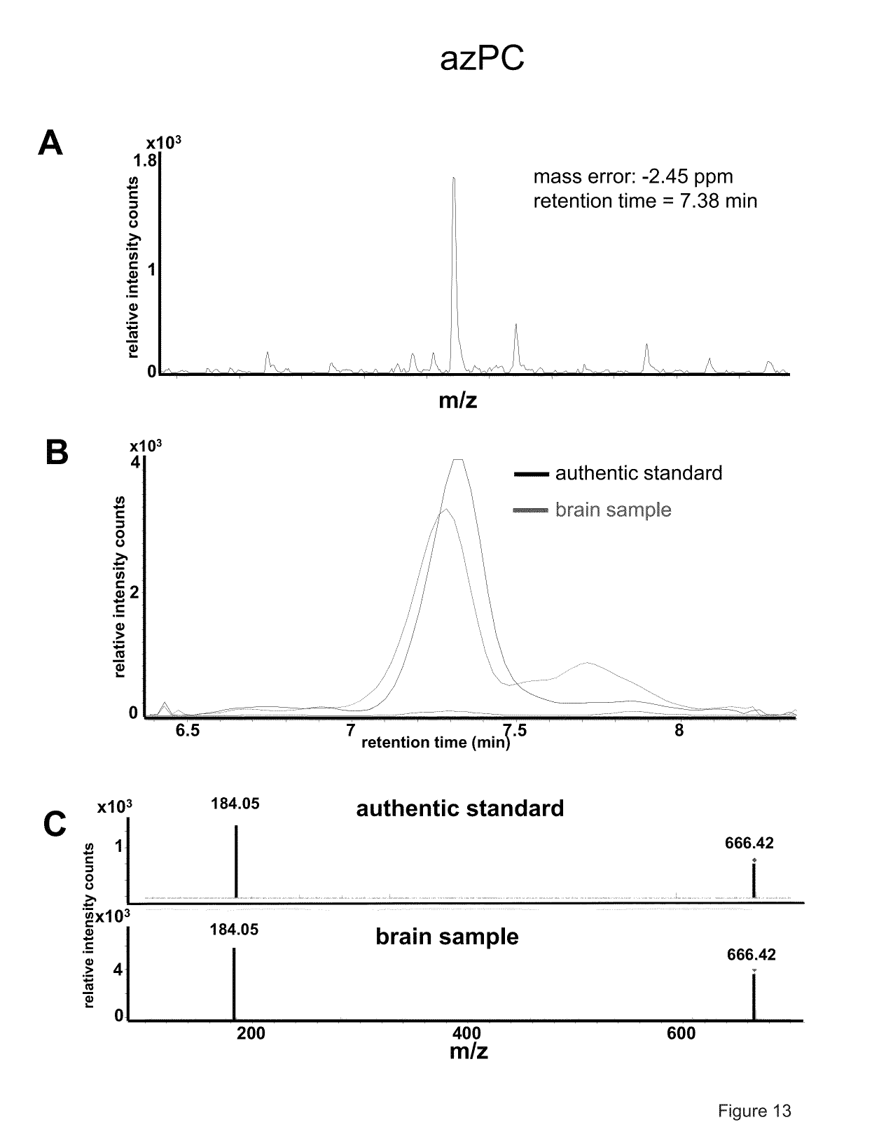

FIG. 14. azPC ester is present in human brain. LC-HRMS detection of azPC ester in human brain sample obtained at autopsy. (A) Accurate mass agreement with calculated mass, (B) retention time on the column in comparison to authentic standards, and (C) MS/MS fragmentation patterns in comparison to authentic standards.

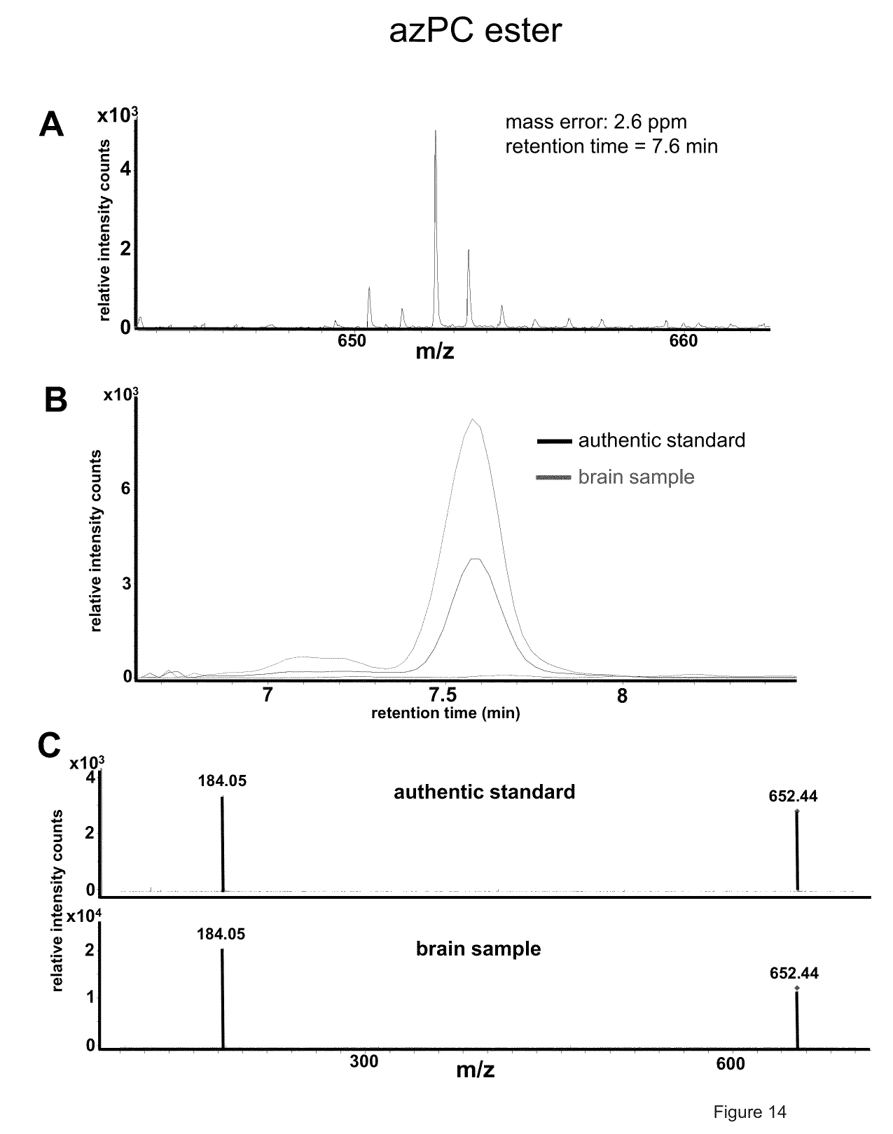

FIG. 15. Oxidized phospholipids suppress proliferation of autoreactive T cells. Proliferation of naive splenocytes stimulated for 48 hours with MBPAc1-11 in the presence of 30 μg/ml of PGPC or lipids related to PGPC: (A) other fatty side-chain derivatives, or (B) other head-group derivatives. *P<0.05 by Student's t-test. Values are the mean±s.e.m. of triplicates. Results are representative of 2 independent experiments.

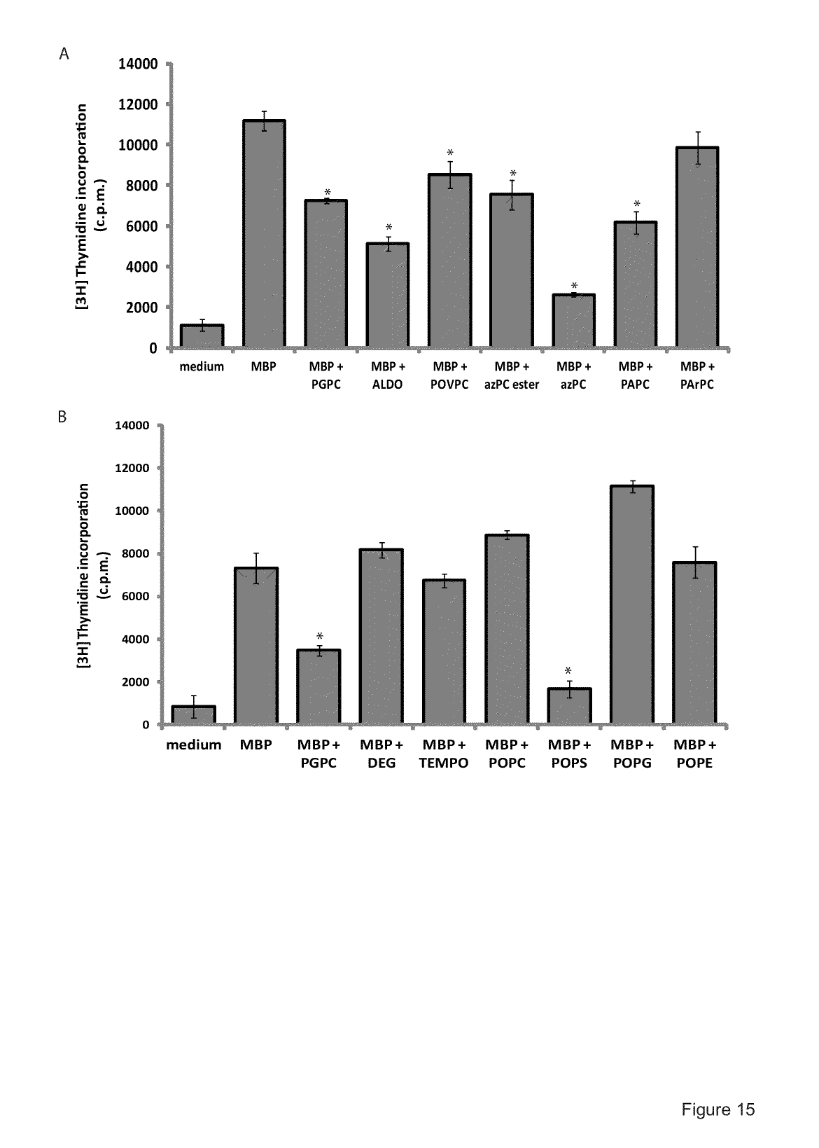

FIG. 16. PGPC, azPC ester, azPC, and POPS suppress PLP139-151-induced cytokine production and proliferation of splenocytes. (A) Cytokine production and (B) proliferation of splenocytes isolated from PLP139-151-immunized SJL mice (10 days after immunization) and re-stimulated in vitro with PLP139-151 in the presence of 30 μg/ml of lipids for 48 hours. *P<0.05 by Student's t-test. Values are the mean±s.e.m. of triplicates. Results are representative of 2 independent experiments.

FIG. 17. POPS, PGPC, azPC ester, and azPC suppress T-cell proliferation independently of CD1d. Proliferation of T cells isolated from splenocytes of wild-type (wt) and CD1d-deficient (Cd1d−/−) mice and stimulated for 48 hours with (A) anti-CD3 or (B) anti-CD3 and anti-CD28 in the presence of 30 μg/ml of POPS, PGPC, azPC ester, or azPC. Values are the mean±s.e.m. *P<0.05 for comparisons made by Student's t-test between each lipid treatment and vehicle treatment of wt cells.

FIG. 18. POPS, PGPC, azPC ester, and azPC suppress proliferation and induce apoptosis of macrophages. (A) Proliferation and (B) apoptosis (annexinV and 7AAD staining) of RAW 264.7 cells stimulated with 100 ng/ml of lipopolysaccharide (LPS) for 48 hours in the presence of 30 μg/ml of lipid. *P<0.05 by Student's t-test comparing each lipid treatment to treatment with LPS alone. Values are the mean±s.e.m. of triplicates. Results are representative of 2 independent experiments.

FIG. 19. POPS, PGPC, azPC ester, and azPC have differential effects on proliferation and apoptosis of naive B cells. (A) Proliferation and (B) apoptosis (annexinV and 7AAD staining) of B cells isolated from spleens of naive mice and stimulated with soluble anti-IgM F(ab′)2 fragment antibody (5 μg/ml) and anti-CD40 antibody (5 μg/ml) for 48 hours in the presence of 30 μg/ml of lipid. *P<0.05 by Student's t-test comparing each lipid treatment to treatment with anti-IgM and anti-CD40 antibodies alone. Values are the mean±s.e.m. of triplicates. Results are representative of 2 independent experiments.

FIG. 20. Phospholipase inhibitor effect on azPC-mediated inhibition of MBPAc1-11 specific T cell proliferation. Select phospholipase inhibitors that had no effect (A) or inhibited the potency of azPC (B) on MBPAc1-11 specific T cell proliferation. Naive MBPAc1-11 splenocytes were pre-incubated with each phospholipase inhibitor or vehicle for 40 minutes prior to activation with 2 μg/ml MBPAc1-11 for 48 hours in the presence of 30 μg/ml azPC. Proliferation response for MBP+azPC is the ratio between the average of triplicate wells for MBP+azPC divided by the average of triplicate wells for MBP.

FIG. 21. Patient demographics and clinical characteristics for cerebrospinal fluid samples presented in FIGS. 1 and 2.

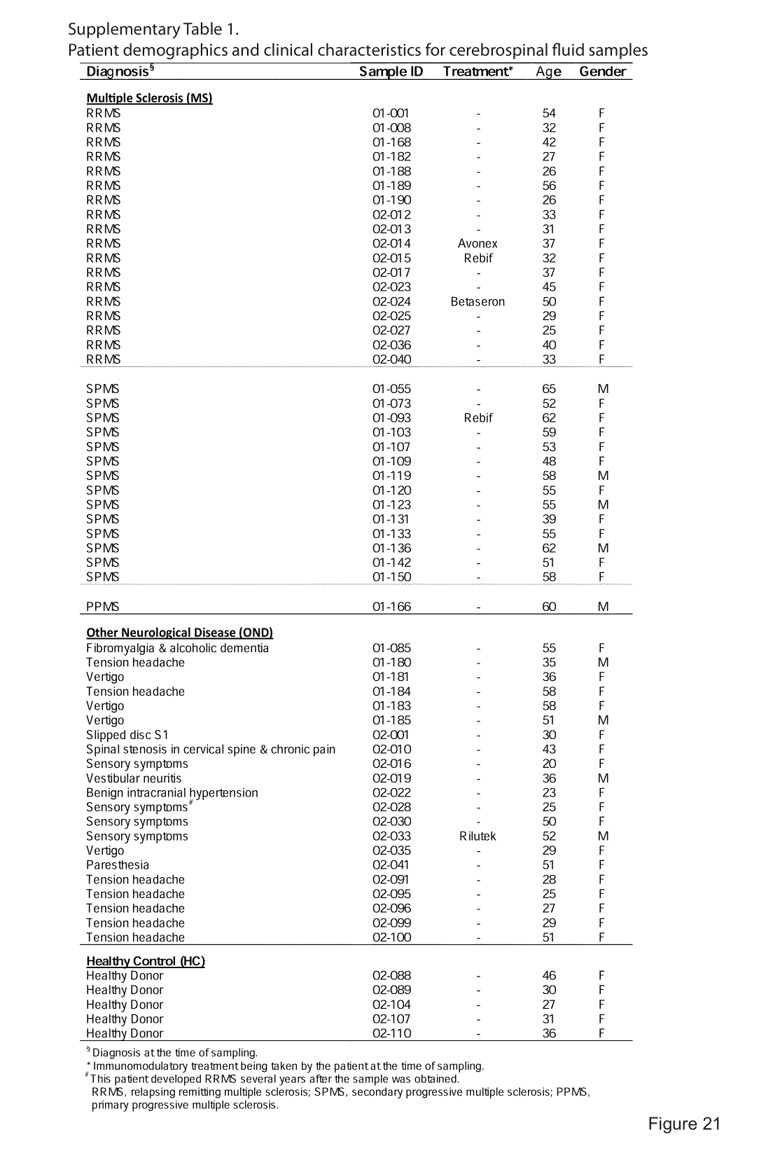

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

The methods of the invention may be specifically applied to individuals that have been diagnosed with an autoimmune disease, e.g. a chronic/progressive or relapsing-remitting disease such as MS or EAE. Treatment is aimed at the treatment or prevention of relapses, which are an exacerbation of a pre-existing condition.

Lipids are fatty acid esters, a class of water-insoluble organic molecules. Lipids consist of a polar or hydrophilic head and one to three nonpolar or hydrophobic tails. The hydrophobic tail consists of one to four fatty acids. These are usually unbranched hydrocarbon chains which may be saturated or unsaturated, although branch-chain sphingoid bases have been described. The chains are usually 14-24 carbon groups long. Biologically relevant lipids are often glycolipids, phospholipids, or sterols. In glycolipids, the head group comprises an oligosaccharide of from 1 to 15 saccharide residues. Phospholipids comprise a negatively charged phosphate group. Sterol head groups comprise a planar steroid ring, for example, cholesterol.

Glycolipids comprise a lipid and saccharide group, which may be a hexose or a pentose, and may be a mono-, di-, tri-, oligo, or polysaccharide, or a derivative thereof. Sugars of interest include allose, altrose, glucose, mannose, gulose, idose, galactose, talose, fructose, maltose, lactose, and sucrose. The linkage between the sugar and the lipid may be at any of the O atoms, and the linkage may be in the alpha or beta configuration.

Lipids of interest include, inter alia, ceramides; gangliosides; cerebrosides, sphingosines; sulfatide; sphingomyelin; phosphatidylamines and phosphatidyl alcohols, such as phosphatidylinositol, phosphatidylserine, phosphatidylcholine, etc.; lipopolysaccharides; LDLs, cholesterols; and the like. Oxidized forms of lipids are of interest, e.g. in the profiling of atherosclerosis, including oxidized or non-oxidized lipids present in serum such as LDLs, and demyelinating diseases, including myelin derived lipids. Lipids may be autoantigens; or may be other lipids of interest for various purposes. Where the lipids are antigens, the antigens may comprise one or more epitopes.

Immune related diseases include: autoimmune diseases in which the immune response aberrantly attacks self-antigens, examples of which include but are not limited to multiple sclerosis (MS), acute disseminated encephalomyelitis (ADEM), rheumatoid arthritis (RA), type I autoimmune diabetes (IDDM), atherosclerosis, systemic lupus erythematosus (SLE), anti-phospholipid antibody syndrome, Guillain-Barre syndrome (GBS) and its subtypes acute inflammatory demyelinating polyradiculoneuropathy, and the autoimmune peripheral neuropathies; allergic diseases in which the immune system aberrantly attacks molecules such as pollen, dust mite antigens, bee venom, peanut oil and other foods, etc.; and tissue transplant rejection in which the immune system aberrantly attacks antigens expressed or contained within a grafted or transplanted tissue, such as blood, bone marrow cells, or solid organs including hearts, lungs, kidneys and livers; and the immune response against tumors. Samples are obtained from patients with clinical symptoms suggestive of an immune-related disease or with an increased likelihood for developing such a disease based on family history or genetic testing.

### Therapeutic Methods

Lipids, including disease associated lipids or analogs thereof, are used to reduce inflammatory responses and/or induce tolerance in a patient. Analogs of interest, without limitation, include those analogs that have altered length and/or saturation of the hydrophobic tail region. Other analogs of interest include those that have altered carbohydrate head groups, e.g. different saccharides; additional heterogroups; and altered stereochemistry, such as different alpha or beta linkage of the saccharide to the lipid; and the like. Candidate analogs may be tested for immune reactivity with any of the methods described herein.

In some embodiments the therapeutic lipid has the structure set forth in Formula I:

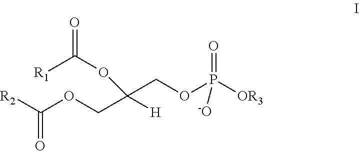

where R₁ and R₂ are independently selected from a linear or branched C₃-C₁₀₀ alkyl; preferably a C₁-C₃₀ alkyl optionally substituted with halo, hydroxy, alkoxy, amino, alkylamino, dialkylamino, sulfate, or phosphate, and which may by saturated, or mono- or di-unsaturated, e.g. 18:0, 24:0 and 24:1. In some embodiments R₁ or R₂ is (Z)-octadec-9-ene. In some embodiments R₂ is hexadecane.

R₃ is selected from H, —CH₂CH₂NH₃ (ethan-1-amine), and serine (2-aminobutanoic acid).

R₄ is absent, or when R₃ is H, R₄ may be

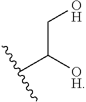 [SMILES: *C(O)CO]

In some embodiments the therapeutic lipid has a structure as set forth below in formulas II-V, where R₁ and R₂ are as defined above.

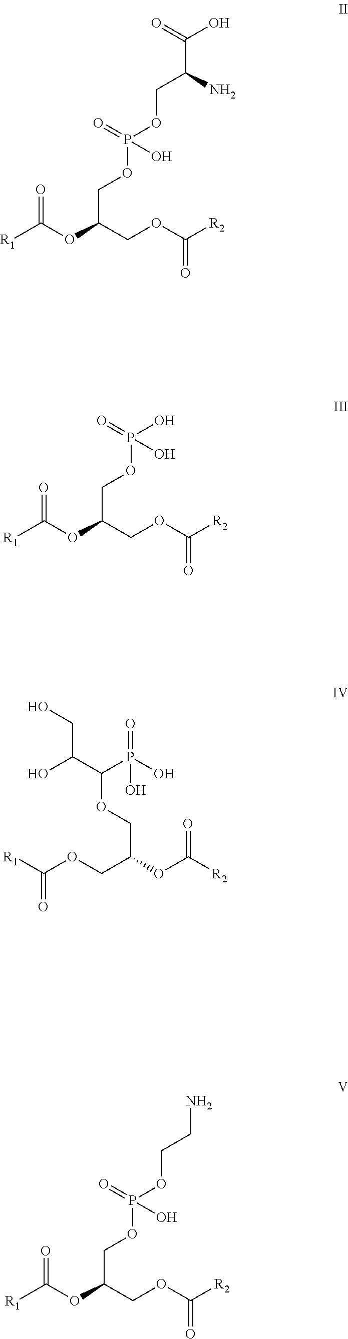

Certain lipids of interest comprise (i) a polar phosphate head group, and the fatty acids (ii) oleate and (iii) palmitate. Examples include (R)-1-(palmitoyloxy)-3-(phosphonooxy)propan-2-yl oleate (POPA); 1-palmitoyl-2-oleoyl-sn-glycero-3-[phospho-1-serine] (POPS), 1-palmitoyl-2-oleoyl-sn-glycero-3-phosphoethanolamine (POPE); 1-palmitoyl-2-oleoyl-sn-glycero-3-phosphoglycerol (POPG), etc.

Also of interest as a therapeutic agent are fatty acids, e.g., including without limitation palmitic acid, ethyl palmitate, sebacic acid, octanoic acid, methyl octanoate, and suberic acid.

The lipid or fatty acid may be administered to a patient to induce tolerance and/or reduce inflammatory responses. As the effectiveness may vary between lipids/fatty acids, the candidate lipid or fatty acid may be tested for suitability. Methods for assessment include administration of a candidate lipid or fatty acid to an animal model for the disease. For example, EAE is demonstrated herein to provide a model for lipid/fatty acid reactivity in multiple sclerosis; a rabbit model for GBS and related peripheral neuropathies is described by Yuki et al. (2001) Annals of Neurology 49:712-720; autoantibodies to oxidized LDL in a rabbit model are described by Nágila et al. (2000) Journal of Nutrition 130:2641-2647; and the like. The candidate lipid or fatty acid is administered to the animal in a tolerizing dose and regimen, and the effect on the disease is measured.

Candidate lipids and fatty acids may also be tested in an in vitro method. Immune cells, e.g. T cells and antigen presenting cells; lymph node cells; bulk splenocytes; peripheral blood lymphocytes; etc. from a patient are contacted with the candidate lipid or fatty acid, and the effect on the cells is determined. Where the lipid has a tolerizing effect and/or reduces the inflammatory response, the immune cells will respond with decreased production of pro-inflammatory cytokines, e.g. γ-IFN; TNFα, etc. Where a lipid or fatty acid has an immunogenic effect, an increased production of pro-inflammatory cytokines is observed.

Therapeutic lipid compositions comprise an immunologically effective amount of lipid, as well as any other compatible components, as needed. By “immunologically effective amount” is meant that the administration of that amount to an individual, either in a single dose or as part of a series, is effective for treatment or prevention of inflammatory/autoimmune disease. This amount varies depending upon the health and physical condition of the individual to be treated, age, individual to be treated (e.g., non-human primate, primate, etc.), the capacity of the individual's immune system, the degree of protection desired, the formulation, the treating clinician's assessment of the medical situation, and other relevant factors. It is expected that the amount will fall in a relatively broad range that can be determined through routine trials. Dosage treatment may be a single dose schedule or a multiple dose schedule (e.g., including booster doses). The therapeutic lipid may be administered in conjunction with other immunoregulatory agents or tolerance-promoting adjuvants.

The effective dose may be empirically determined using animal models and in vitro models, and the dose will depend at least in part on the route of administration. The lipids may be administered orally, in an aerosol spray; by injection, e.g. i.m., s.c., i.p., i.v., etc.

The therapeutic lipid or fatty acid may be administered in a single dose, or in multiple doses, usually multiple doses over a period of time, e.g. weekly, semi-weekly, monthly etc. for a period of time sufficient to reduce severity of the autoimmune disease, which may comprise 1, 2, 3, 4, 6, 10, or more doses. Dosing may be from 1 to 7 times weekly, for example daily, every other day, every third day, semi-weekly, weekly. In some embodiments administration is every other day, i.e. about every 48 hours.

The lipid or fatty acid dose may be from about 0.01 mg/kg patient weight; about 0.1 mg/kg patient weight; about 1 mg/kg; about 10 mg/kg; to about 100 mg/kg. The lipid dose will usually not exceed about 100 mg/kg, and usually not exceed 10 mg/kg.

The lipid or fatty acid therapeutic compositions are administered in a pharmaceutically acceptable excipient, e.g. a lipid based solution or emulsion. The term “pharmaceutically acceptable” refers to an excipient acceptable for use in the pharmaceutical and veterinary arts, which is not toxic or otherwise inacceptable. Examples of suitable lipid-based excipients include mono-, di- and tri-glycerides, especially naturally extracted unsaturated edible oils in hydrogenated form (such as vegetable oil, castor oil, cottonseed oil, corn oil, canola oil, rapeseed oil, peanut oil, sesame seed oil, coconut oil and mixtures thereof). The lipid may be administered in a detergent solution, e.g. 0.1 to 1% Tween-20, etc.

The compositions may also include a tolerance-promoting adjuvant. A tolerance-promoting adjuvant is a pharmacological or immunological agent that is provided with an antigen to enhance the recipient's immune response and tolerance to the antigen. Examples of known agents that can be combined with therapeutic lipids or fatty acidsto reduce inflammation and enhance tolerance induction include: (i) interleukins such as IL-4, IL-10, IL-13, TGFbeta and other cytokines and/or chemokines that promote tolerance; (ii) immunoinhibitor oligonucleotide sequences, such as GpG-oligonucleotides (Ho P P et al, Journal Immunology, 175(9):6226-34, 2005); (iii) small molecules identified to promote immune tolerance such as statin drugs (Youssef S, Nature, 20(6911):78-84, 2002), anti-histamines (Pedotti et al, Proc. Natl. Acad. Sci. USA, 100(4):1867-72, 2003), tryptophan metabolites (Platten M et al, Science, 310: 850-5, 2005). The effectiveness of a tolerance-promoting adjuvant may be determined by measuring the T and B cell responses against the lipid antigen as described below for sulfatide, sphingomyelin and PGPC.

The therapeutic lipid or fatty acid may be combined with conventional excipients, such as pharmaceutical grades of mannitol, lactose, starch, magnesium stearate, sodium saccharin, talcum, cellulose, glucose, sucrose, magnesium, carbonate, Tween-20, dimethylsulfoxide (DMSO), and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of tolerogen in these formulations can vary widely, and will be selected primarily based on fluid volumes, viscosities, body weight and the like in accordance with the particular mode of administration selected and the patient's needs. The resulting compositions may be in the form of a solution, suspension, tablet, pill, capsule, powder, gel, cream, lotion, ointment, aerosol or the like.

The concentration of therapeutic lipid or fatty acid of the invention in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

Treating, treatment, or therapy of a disease or disorder shall mean slowing, stopping or reversing the disease's progression by administration of a lipid or lipids. In the preferred embodiment, treating a disease means reversing the disease's progression, ideally to the point of eliminating the disease itself. As used herein, ameliorating a disease and treating a disease are equivalent.

Preventing, prophylaxis or prevention of a disease or disorder as used in the context of this invention refers to the administration of a lipid or lipids to prevent the occurrence or onset of a disease or disorder or some or all of the symptoms of a disease or disorder or to lessen the likelihood of the onset of a disease or disorder.

Therapeutic administration of lipids or fatty acids can be used to both prevent the onset of inflammation and autoimmune diseases, and to treat inflammatory and autoimmune disease. For the treatment of established autoimmune disease, patients with the clinical diagnosis of multiple sclerosis, peripheral neuropathies, systemic lupus erythematosus or another autoimmune disease targeting lipids, are administered lipid or fatty acid in a tolerizing regimen to reduce the symptoms, severity and/or clinical progression of the disease. For certain autoimmune diseases, biomarkers have been identified that predict which asymptomatic or early-symptomatic individuals will progress to develop definite autoimmune disease. Such biomarkers can include genetic, protein and/or lipid molecules. For example, in patients with clinically isolated syndrome the presence of autoantibodies targeting myelin oligodendrocyte glycoprotein (MOG) and/or myelin basic protein (MBP) predict an increased likelihood for progression to clinically definite multiple sclerosis (Berger et al, New England Journal of Medicine, 349(2):139-45, 2003).

Thus, in addition to the treatment of patients with established inflammation and/or autoimmunity, therapeutic administration of lipids can be used to prevent the development of inflammation and/or autoimmunity in asymptomatic or early symptomatic individuals for which testing for genetic, protein and/or lipid biomarkers predicts progression to a clinical autoimmune disease.

### Conditions for Analysis and Therapy

The compositions and methods of the invention find use in combination with a variety of conditions. Among these are autoimmune diseases having a lipid component. It has been found that demyelinating autoimmune diseases, in particular, have a lipid component, as does IDDM, SLE, coronary artery disease, etc.

Demyelinating diseases may be characterized according to the presence of autoantibodies specific for lipids associated with the nervous system, and in particular with myelin. Myelin sheaths, which cover many nerve fibers, are composed of lipoprotein layers formed in early life. Myelin formed by the oligodendroglia in the CNS differs chemically and immunologically from that formed by the Schwann cells peripherally, but both types have the same function: to promote transmission of a neural impulse along an axon. Demyelinating diseases include those that affect the central nervous system, and those that affect the peripheral nervous system. CNS conditions include multiple sclerosis, acute disseminated encephalomyelitis (ADEM), neuromyelitis optica (NMO), and the animal model EAE, which are progressive CNS diseases characterized by disseminated patches of demyelination, resulting in multiple and varied neurologic symptoms and signs, usually with remissions and exacerbations.

Plaques of demyelination, with destruction of oligodendroglia and perivascular inflammation, are disseminated throughout the CNS, primarily in the white matter, with a predilection for the lateral and posterior columns (especially in the cervical and dorsal regions), the optic nerves, and periventricular areas. Tracts in the midbrain, pons, and cerebellum are also affected as is gray matter in the cerebrum and spinal cord. Cell bodies and axons are usually preserved, especially in recent lesions. Later, axons may be destroyed, especially in the long tracts, and a fibrous gliosis makes the tracts appear sclerotic. Recent and old lesions may coexist. Chemical changes in lipid and protein constituents of myelin occur in and around the plaques.

Multiple sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive (primary progressive MS, PPMS). Relapsing remitting MS (RR MS) is characterized clinically by relapses and remissions that occur over months to years, with partial or full recovery of neurological deficits between attacks. Such patients manifest approximately 1 attack, or relapse, pre year. Over 10 to 20 years, approximately 50% of RR MS patients develop secondary progressive MS (SP MS) which is characterized by incomplete recovery between attacks and accumulation of neurologic deficits resulting in increasing disability.

Diagnosis is indirect, by deduction from clinical, radiographic (brain plaques on magnetic resonance [MR] scan), and to a lesser extent laboratory (oligoclonal bands on CSF analysis) features. Typical cases can usually be diagnosed confidently on clinical grounds. The diagnosis can be suspected after a first attack. Later, a history of remissions and exacerbations and clinical evidence of CNS lesions disseminated in more than one area are highly suggestive.

MRI, the most sensitive diagnostic imaging technique, may show plaques. It may also detect treatable nondemyelinating lesions at the junction of the spinal cord and medulla (eg, subarachnoid cyst, foramen magnum tumors) that occasionally cause a variable and fluctuating spectrum of motor and sensory symptoms, mimicking MS. Gadolinium-contrast enhancement can distinguish areas of active inflammation from older brain plaques. MS lesions may also be visible on contrast-enhanced CT scans; sensitivity may be increased by giving twice the iodine dose and delaying scanning (double-dose delayed CT scan).

Treatments for MS include interferon β (Avonex, Betaseron, Rebif), Copaxone (Glatiramer acetate), and anti-VLA4 (Tysabri, natalizumab), which reduce relapse rate and to date have only exhibited a modest impact on disease progression. MS is also treated with immunosuppressive agents including methylprednisolone, other steroids, methotrexate, cladribine and cyclophosphamide. Many biological agents, such as anti-IFNgamma antibody, CTLA4-Ig (Abetacept), anti-CD20 (Rituxan), and other anti-cytokine agents are in clinical development for MS.

Conventional treatments for MS include interferon 13 (Avonex, Betaseron, Rebif), Copaxone (Glatiramer acetate), and anti-VLA4 (Tysabri, natalizumab), which reduce relapse rate and to date have only exhibited a modest impact on disease progression. MS is also treated with immunosuppressive agents including methylprednisolone, other steroids, methotrexate, cladribine and cyclophosphamide. Many biological agents, such as anti-IFNgamma antibody, CTLA4-Ig (Abetacept), anti-CD20 (Rituxan), and other anti-cytokine agents are in clinical development for MS.

Neuromyelitis optica (NMO), or Devic's disease, is an autoimmune, inflammatory disorder of the optic nerves and spinal cord. Although inflammation may affect the brain, the disorder is distinct from multiple sclerosis, having a different pattern of response to therapy, possibly a different pattern of autoantigens and involvement of different lymphocyte subsets.

The main symptoms of Devic's disease are loss of vision and spinal cord function. As for other etiologies of optic neuritis, the visual impairment usually manifests as decreased visual acuity, although visual field defects, or loss of color vision may occur in isolation or prior to formal loss of acuity. Spinal cord dysfunction can lead to muscle weakness, reduced sensation, or loss of bladder and bowel control. The damage in the spinal cord can range from inflammatory demyelination to necrotic damage of the white and grey matter. The inflammatory lesions in Devic's disease have been classified as type II lesions (complement mediated demyelinization), but they differ from MS pattern II lesions in their prominent perivascular distribution. Therefore, the pattern of inflammation is often quite distinct from that seen in MS.

Attacks are conventionally treated with short courses of high dosage intravenous corticosteroids such as methylprednisolone IV. When attacks progress or do not respond to corticosteroid treatment, plasmapheresis may be used. Commonly used immunosuppressant treatments include azathioprine (Imuran) plus prednisone, mycophenolate mofetil plus prednisone, Rituximab, Mitoxantrone, intravenous immunoglobulin (IVIG), and Cyclophosphamide. The monoclonal antibody rituximab is under study.

The disease can be monophasic, i.e. a single episode with permanent remission. However, at least 85% of patients have a relapsing form of the disease with repeated attacks of transverse myelitis and/or optic neuritis. In patients with the monophasic form the transverse myelitis and optic neuritis occur simultaneously or within days of each other. On the other hand, patients with the relapsing form are more likely to have weeks or months between the initial attacks and to have better motor recovery after the initial transverse myelitis event. Relapses usually occur early with about 55% of patients having a relapse in the first year and 90% in the first 5 years. Unlike MS, Devic's disease rarely has a secondary progressive phase in which patients have increasing neurologic decline between attacks without remission. Instead, disabilities arise from the acute attacks.

Acute disseminated encephalomyelitis (ADEM) is an immune mediated disease of the brain that can occur spontaneously, or following a viral infection, vaccination, bacterial or parasitic infection. It is considered part of the Multiple sclerosis borderline diseases. The incidence rate is about 8 per 1,000,000 people per year. Although it occurs in all ages, most reported cases are in children and adolescents, with the average age around 5 to 8 years old. The mortality rate may be as high as 5%, full recovery is seen in 50 to 75% of cases, while up to 70 to 90% recover with some minor residual disability. The average time to recover is one to six months.

ADEM produces multiple inflammatory lesions in the brain and spinal cord, particularly in the white matter, i.e. demyelination. Usually these are found in the subcortical and central white matter and cortical gray-white junction of both cerebral hemispheres, cerebellum, brainstem, and spinal cord, but periventricular white matter and gray matter of the cortex, thalami and basal ganglia may also be involved.

When the patient suffers more than one demyelinating episode, it may be referred to as recurrent disseminated encephalomyelitis or multiphasic disseminated encephalomyelitis (MDEM). Acute hemorrhagic leukoencephalitis (AHL, or AHLE), also known as acute necrotizing encephalopathy (ANE), acute hemorrhagic encephalomyelitis (AHEM), acute necrotizing hemorrhagic leukoencephalitis (ANHLE), Weston-Hurst syndrome, or Hurst's disease, is a hyperacute and frequently fatal form of ADEM, and is characterized by necrotizing vasculitis of venules and hemorrhage, and edema. Death is common in the first week and overall mortality is about 70%, but increasing evidence points to favorable outcomes after aggressive treatment with corticosteroids, immunoglobulins, cyclophosphamide, and plasma exchange.

Peripheral neuropathies include Guillain-Barre syndrome (GBS) with its subtypes acute inflammatory demyelinating polyradiculoneuropathy, acute motor axonal neuropathy, acute motor and sensory axonal neuropathy, Miller Fisher syndrome, and acute pandysautonomia; chronic inflammatory demyelinating polyneuropathy (CIDP) with its subtypes classical CIDP, CIDP with diabetes, CIDP/monoclonal gammopathy of undetermined significance (MGUS), sensory CIDP, multifocal motor neuropathy (MMN), multifocal acquired demyelinating sensory and motor neuropathy or Lewis-Sumner syndrome, multifocal acquired sensory and motor neuropathy, and distal acquired demyelinating sensory neuropathy; IgM monoclonal gammopathies with its subtypes Waldenstrom's macroglobulinemia, myelin-associated glycoprotein-associated gammopathy, polyneuropathy, organomegaly, endocrinopathy, M-protein, skin changes syndrome, mixed cryoglobulinemia, gait ataxia, late-onset polyneuropathy syndrome, and MGUS.

SLE.

Systemic lupus erythematosus (SLE) is an autoimmune disease characterized by polyclonal B cell activation, which results in a variety of anti-protein and non-protein autoantibodies (see Kotzin et al. (1996) Cell 85:303-306 for a review of the disease). These autoantibodies form immune complexes that deposit in multiple organ systems, causing tissue damage. SLE is a difficult disease to study, having a variable disease course characterized by exacerbations and remissions. For example, some patients may demonstrate predominantly skin rash and joint pain, show spontaneous remissions, and require little medication. The other end of the spectrum includes patients who demonstrate severe and progressive kidney involvement (glomerulonephritis) that requires therapy with high doses of steroids and cytotoxic drugs such as cyclophosphamide.

Multiple factors may contribute to the development of SLE. Several genetic loci may contribute to susceptibility, including the histocompatibility antigens HLA-DR2 and HLA-DR3. The polygenic nature of this genetic predisposition, as well as the contribution of environmental factors, is suggested by a moderate concordance rate for identical twins, of between 25 and 60%.

Many causes have been suggested for the origin of autoantibody production. Proposed mechanisms of T cell help for anti-dsDNA antibody secretion include T cell recognition of DNA-associated protein antigens such as histones and recognition of anti-DNA antibody-derived peptides in the context of class II MHC. The class of antibody may also play a factor. In the hereditary lupus of NZB/NZW mice, cationic IgG2a anti-double-stranded (ds) DNA antibodies are pathogenic. The transition of autoantibody secretion from IgM to IgG in these animals occurs at the age of about six months, and T cells may play an important role in regulating the IgG production.

Disease manifestations result from recurrent vascular injury due to immune complex deposition, leukothrombosis, or thrombosis. Additionally, cytotoxic antibodies can mediate autoimmune hemolytic anemia and thrombocytopenia, while antibodies to specific cellular antigens can disrupt cellular function. An example of the latter is the association between anti-neuronal antibodies and neuropsychiatric SLE.

Atherosclerotic plaque consists of accumulated intracellular and extracellular lipids, smooth muscle cells, connective tissue, and glycosaminoglycans. The earliest detectable lesion of atherosclerosis is the fatty streak, consisting of lipid-laden foam cells, which are macrophages that have migrated as monocytes from the circulation into the subendothelial layer of the intima, which later evolves into the fibrous plaque, consisting of intimal smooth muscle cells surrounded by connective tissue and intracellular and extracellular lipids.

Interrelated hypotheses have been proposed to explain the pathogenesis of atherosclerosis. The lipid hypothesis postulates that an elevation in plasma LDL levels results in penetration of LDL into the arterial wall, leading to lipid accumulation in smooth muscle cells and in macrophages. LDL also augments smooth muscle cell hyperplasia and migration into the subintimal and intimal region in response to growth factors. LDL is modified or oxidized in this environment and is rendered more atherogenic. The modified or oxidized LDL is chemotactic to monocytes, promoting their migration into the intima, their early appearance in the fatty streak, and their transformation and retention in the subintimal compartment as macrophages. Scavenger receptors on the surface of macrophages facilitate the entry of oxidized LDL into these cells, transferring them into lipid-laden macrophages and foam cells. Oxidized LDL is also cytotoxic to endothelial cells and may be responsible for their dysfunction or loss from the more advanced lesion.

The chronic endothelial injury hypothesis postulates that endothelial injury by various mechanisms produces loss of endothelium, adhesion of platelets to subendothelium, aggregation of platelets, chemotaxis of monocytes and T-cell lymphocytes, and release of platelet-derived and monocyte-derived growth factors that induce migration of smooth muscle cells from the media into the intima, where they replicate, synthesize connective tissue and proteoglycans, and form a fibrous plaque. Other cells, e.g. macrophages, endothelial cells, arterial smooth muscle cells, also produce growth factors that can contribute to smooth muscle hyperplasia and extracellular matrix production.

Endothelial dysfunction includes increased endothelial permeability to lipoproteins and other plasma constituents, expression of adhesion molecules and elaboration of growth factors that lead to increased adherence of monocytes, macrophages and T lymphocytes. These cells may migrate through the endothelium and situate themselves within the subendothelial layer. Foam cells also release growth factors and cytokines that promote migration of smooth muscle cells and stimulate neointimal proliferation, continue to accumulate lipid and support endothelial cell dysfunction. Clinical and laboratory studies have shown that inflammation plays a major role in the initiation, progression and destabilization of atheromas.

The “autoimmune” hypothesis postulates that the inflammatory immunological processes characteristic of the very first stages of atherosclerosis are initiated by humoral and cellular immune reactions against an endogenous antigen. Human Hsp60 expression itself is a response to injury initiated by several stress factors known to be risk factors for atherosclerosis, such as hypertension. Oxidized LDL (oxLDL) is another candidate for an autoantigen in atherosclerosis. Antibodies to oxLDL have been detected in patients with atherosclerosis, and they have been found in atherosclerotic lesions. T lymphocytes isolated from human atherosclerotic lesions have been shown to respond to oxLDL and to be a major autoantigen in the cellular immune response. A third autoantigen proposed to be associated with atherosclerosis is 2-Glycoprotein I (2GPI), a glycoprotein that acts as an anticoagulant in vitro. 2GPI is found in atherosclerotic plaques, and hyper-immunization with 2GPI or transfer of 2GPI-reactive T cells enhances fatty streak formation in transgenic atherosclerotic-prone mice.

Infections may contribute to the development of atherosclerosis by inducing both inflammation and autoimmunity. A large number of studies have demonstrated a role of infectious agents, both viruses (cytomegalovirus, herpes simplex viruses, enteroviruses, hepatitis A) and bacteria (C. pneumoniae, H. pylori, periodontal pathogens) in atherosclerosis. Recently, a new “pathogen burden” hypothesis has been proposed, suggesting that multiple infectious agents contribute to atherosclerosis, and that the risk of cardiovascular disease posed by infection is related to the number of pathogens to which an individual has been exposed. Of single micro-organisms, C. pneumoniae probably has the strongest association with atherosclerosis.

These hypotheses are closely linked and not mutually exclusive. Modified LDL is cytotoxic to cultured endothelial cells and may induce endothelial injury, attract monocytes and macrophages, and stimulate smooth muscle growth. Modified LDL also inhibits macrophage mobility, so that once macrophages transform into foam cells in the subendothelial space they may become trapped. In addition, regenerating endothelial cells (after injury) are functionally impaired and increase the uptake of LDL from plasma.

Atherosclerosis is characteristically silent until critical stenosis, thrombosis, aneurysm, or embolus supervenes. Initially, symptoms and signs reflect an inability of blood flow to the affected tissue to increase with demand, e.g. angina on exertion, intermittent claudication. Symptoms and signs commonly develop gradually as the atheroma slowly encroaches on the vessel lumen. However, when a major artery is acutely occluded, the symptoms and signs may be dramatic.

Currently, due to lack of appropriate diagnostic strategies, the first clinical presentation of more than half of the patients with coronary artery disease is either myocardial infarction or death. Further progress in prevention and treatment depends on the development of strategies focused on the primary inflammatory process in the vascular wall, which is fundamental in the etiology of atherosclerotic disease.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Identification of Fatty Acids of the Myelin Sheath that Resolve Neuroinflammation

A “functional lipidomics” approach was used to discover autoimmune targets and develop novel therapeutic strategies for MS. We used lipid autoantigen microarrays and lipid mass spectrometry to identify targets of the adaptive autoimmune response in MS patients. We then explored these results in an animal model of MS, experimental autoimmune encephalomyelitis (EAE), in order to define the biological role of the autoantibody-targeted lipids in the pathogenesis of autoimmune demyelination. Unexpectedly, we found that several of the autoantibody-targeted lipids—phospholipids naturally present in the brain—could both prevent and treat EAE. Our findings show that phospholipids containing saturated fatty-acid side chains serve as natural brakes on inflammatory responses in the CNS and that this protective mechanism is compromised in MS, as these guardian lipids are attacked by the adaptive arm of the immune system. These naturally occurring myelin lipids have therapeutic potential in MS and other inflammatory brain diseases.

### Results

Anti-Lipid-Antibody Reactivity Differentiates Between Ms Patients and Controls.

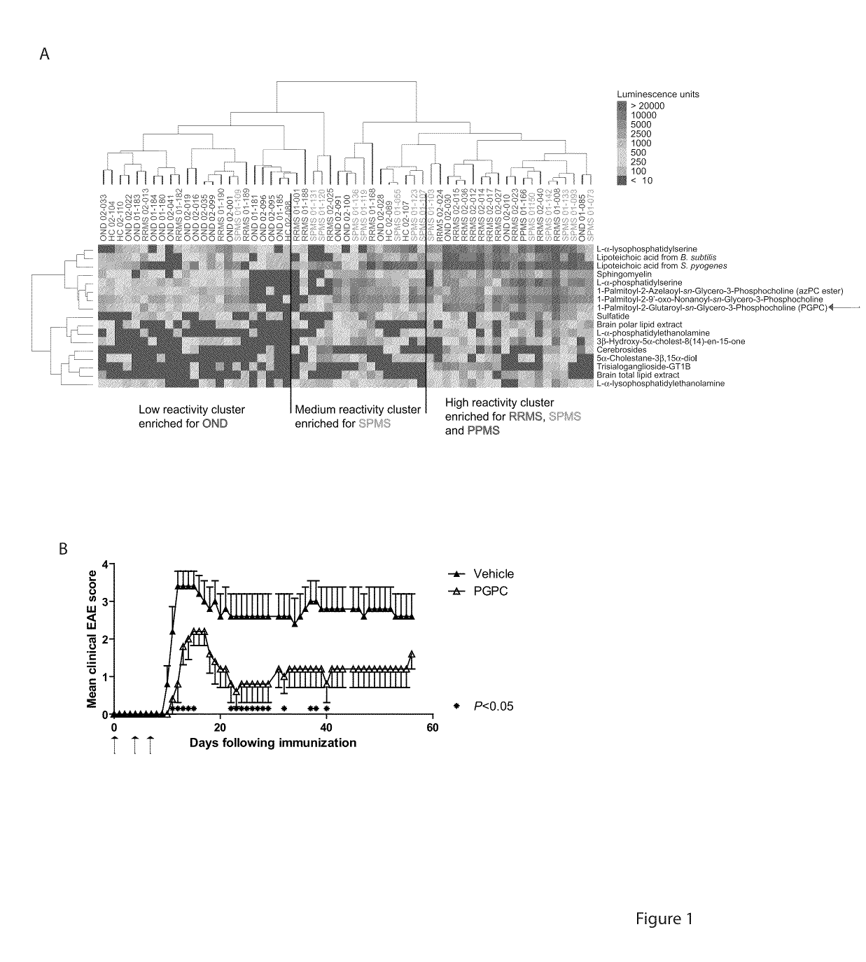

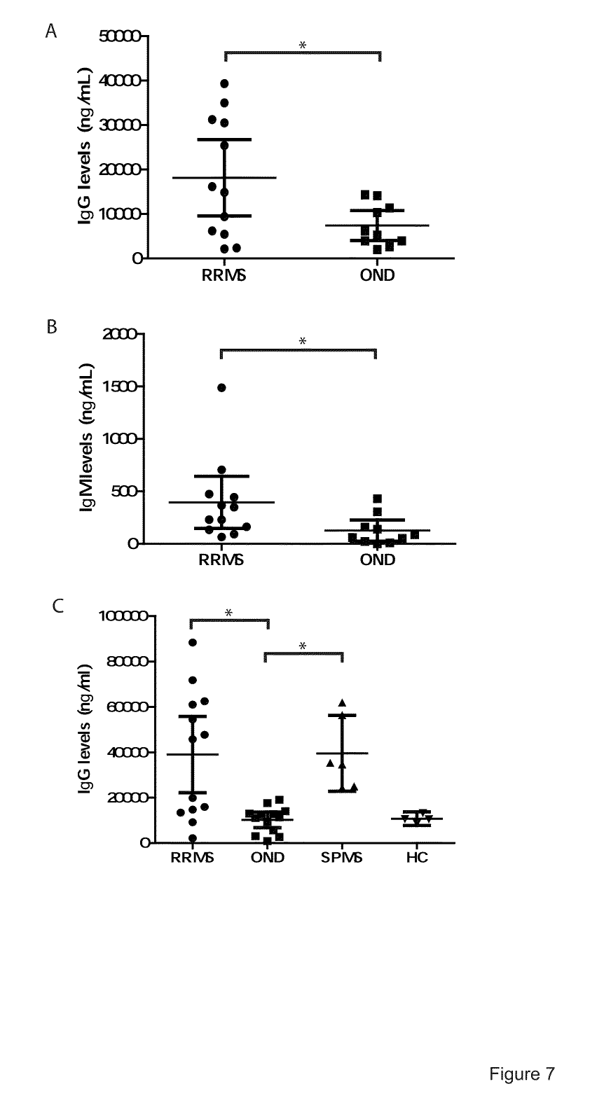

We printed lipid antigen microarrays containing over 50 brain lipids and used these arrays to profile anti-lipid autoantibodies in cerebrospinal fluid (CSF) samples derived from MS and control patients. The Significance Analysis of Microarrays (SAM) algorithm identified 17 lipids that had significantly greater reactivity with autoantibodies in CSF from the 33 individuals with MS (18 with relapsing remitting MS [RRMS], 14 with secondary progressive MS [SPMS], and 1 with primary progressive MS [PPMS]) than in that from the 26 controls (21 with other (non-inflammatory) neurological diseases [OND] and 5 healthy controls [HC]) (false discovery rate [FDR]=0.048). We used a hierarchical cluster algorithm to order the patient samples and SAM-identified lipids. Most MS samples clustered together according to the similarity of their anti-lipid autoantibody profiles (FIG. 1A). Specifically, the PPMS sample, and half of the RRMS and SPMS samples, clustered in the group with the highest anti-lipid autoantibody reactivity, whereas only 3 of 21 the OND and none of the HC samples were represented in this group. Most of the controls (15 of 21 OND and 3 of 5 HC) clustered in the group with the lowest anti-lipid autoantibody reactivity, whereas only one SPMS sample and 4 of the 18 RRMS samples clustered in this group. ELISA analysis showed that levels of total IgG were higher than levels of total IgM in both RRMS and OND CSF (FIG. 7A, B), and that levels of total IgG were significantly higher in RRMS and SPMS CFS than in OND CSF (FIG. 7C).

PGPC Administration Before Disease Onset Reduces EAE Severity.

To determine whether the autoantibody-targeted lipids have a role in autoimmune demyelination, we tested the effect of select lipids on EAE, a mouse model of MS. We initially screened lipids that fell into 4 categories: 1) brain and myelin lipids, e.g. cerebrosides, sulfatides, and gangliosides; 2) membrane lipids, e.g. cholesterol, phosphatidylcholine, and sphingomyelin; 3) oxidized lipids, e.g. 1-Palmitoyl-2-Glutaroyl-sn-Glycero-3-Phosphocholine (PGPC) and its derivatives; and 4) microbial lipids, e.g. LPS and lipoteichoic acid. From our set of 17 anti-lipid autoantibody hits, we selected several lipids from each category that appeared to have higher autoantibody reactivity in MS samples than in OND or HC samples. These were then tested in T-cell proliferation assays and in EAE. We found that cerebrosides and gangliosides were unable to suppress MBP_{Ac1-11}-specific T-cell proliferation, that administration of cerebrosides did not affect EAE, and that oxidized cholesterol had only a minimal effect on EAE. We previously tested sulfatides, which worsened EAE.

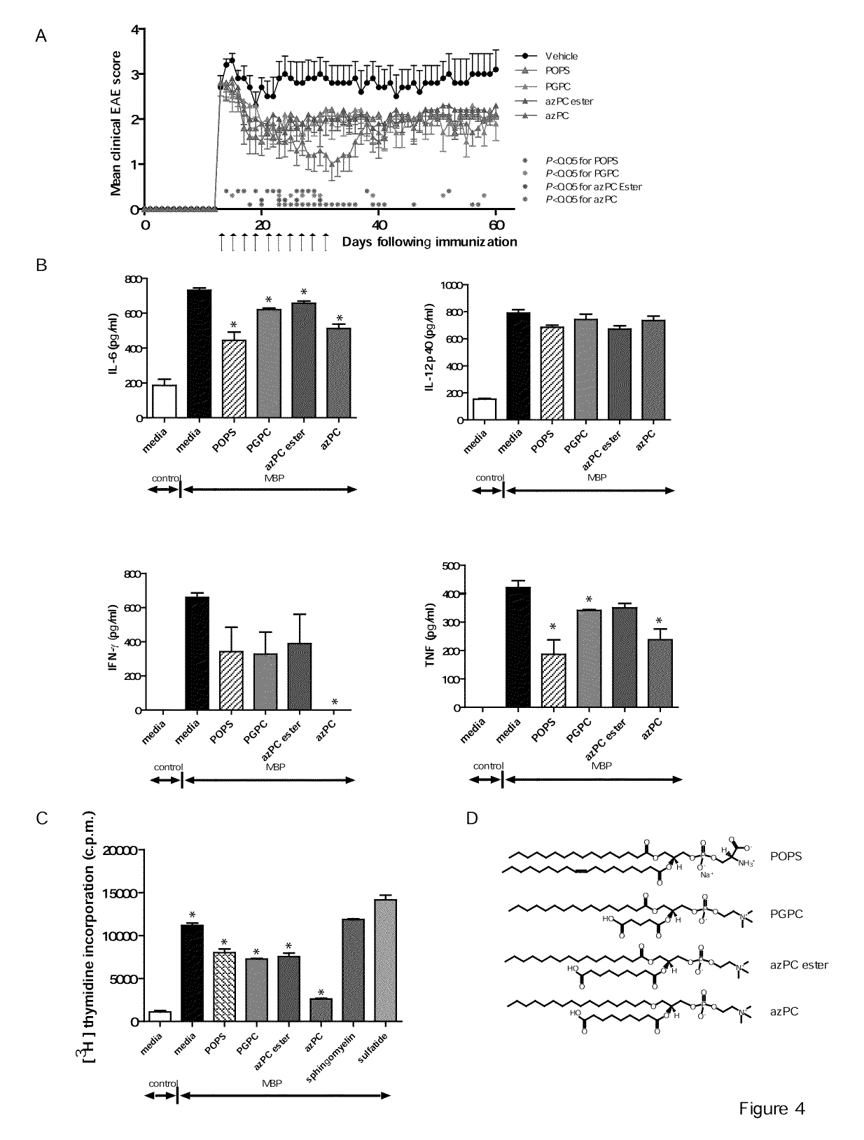

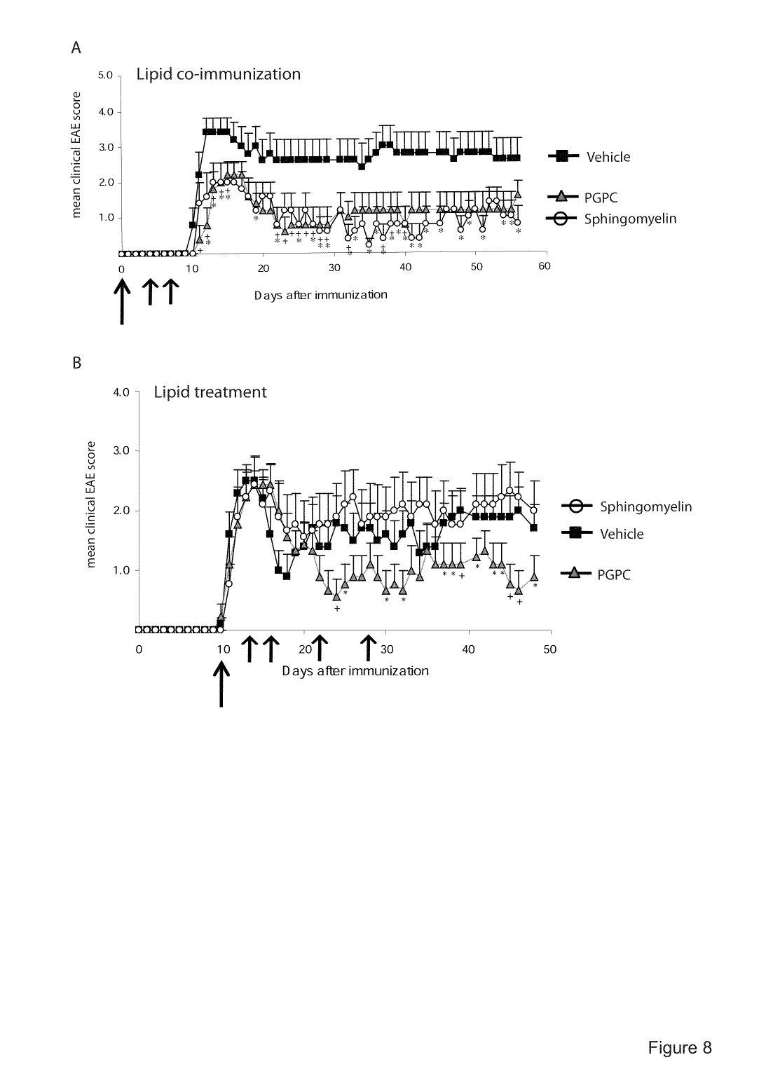

PGPC, one of the oxidized lipids tested in this screen, was of particular interest because it is a derivative of oxidized phosphatidylcholine, which has previously been shown to be targeted by autoantibodies in MS. Indeed, phosphatidylcholine comprises 30.1% of the lipids in the gray matter and 15.0% of the lipids in the white matter of the adult human brain, lipid peroxidation occurs in MS lesions, and oxidized phosphatidylcholine is present in MS lesions. We therefore tested the effect of exogenous PGPC on EAE, using two treatment regimens. In the first regimen, we subcutaneously administered PGPC together with the proteolipid protein (PLP)₁₃₉₋₁₅₁ that we use to induce EAE in to SJL/J mice, and then we intraperitoneally administered PGPC on its own four and seven days after immunization. In contrast to sulfatide, a myelin glycosphingolipid that exacerbated EAE when delivered in this prophylactic regimen, PGPC unexpectedly reduced the severity of EAE throughout the disease course (FIG. 1B and FIG. 8A). In the second regimen, we started administering PGPC 10 days after immunization, i.e. at the time of disease onset (FIG. 8B), and found that PGPC could also attenuated EAE that was already established. Sphingomyelin, which composes 6.9% and 7.7% of the lipids in the gray and white matter, respectively, of an adult human brain, also attenuated EAE when administered during the immunization (FIG. 8A). However, when administered 10 days after the immunization, sphingomyelin exacerbated EAE (FIG. 8B). Thus, unlike the other lipids tested, PGPC attenuated the development of EAE and ameliorated established EAE (FIG. 8 and FIG. 4A).

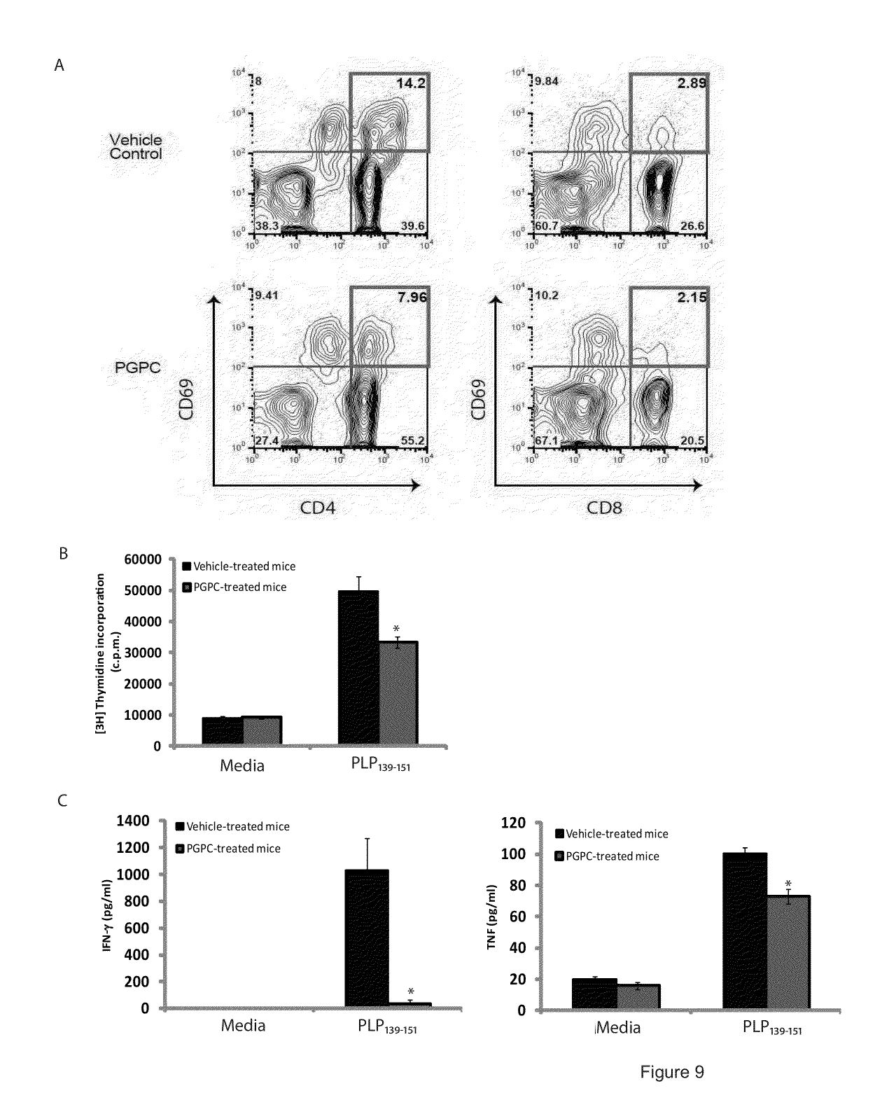

Lymph node cells isolated from EAE mice treated prophylactically with PGPC showed a marked reduction in the expression of the early activation marker CD69 among CD4+ T cells. Specifically, 14.2% of the cells isolated from vehicle-treated mice were CD4+CD69+, compared to 7.96% of the cells isolated from PGPC-treated mice (FIG. 9A). Moreover, compared to cells from vehicle-treated mice, lymph node cells and splenocytes isolated from PGPC-treated mice secreted less IFN— upon stimulation with the encephalitogenic PLP139-151 peptide (FIG. 9B). Thus, a reduction in T-cell activation, a process important in MS pathogenesis, accompanies the PGPC-induced attenuation of EAE.

Phosphocholine Head Group Confers Antigenicity.

To define the basis for autoimmune targeting of PGPC and to identify additional lipids that might modulate EAE, we characterized the lipid components targeted by autoantibodies in MS. The commonality of the phosphatidylcholine backbone in lipids targeted by autoantibodies in MS prompted us to explore this structure as a potential determinant of antigenicity. We investigated autoantibody targeting of 7 lipids that have a glycero-3-phosphocholine backbone in common with PGPC, as well as targeting of other structurally similar lipids from the lipid array used in the previous experiments (FIG. 1A)—such as those containing features in common with PGPC, e.g., a phosphate head group with one or two non-polar side chains. We used Mini Array I, comprising 17 lipids (Table 1), to profile autoantibody responses in CSF samples from RRMS patients and OND controls.

TABLE 1

| | |
|---|---|
| Name | Avanti Lipid Catalog Number |

Mini-Array I

| | |
|---|---|
| 1-Palmitoyl-2-Glutaroyl-sn-Glycero-3-Phosphocholine | 870602 |
| 1-Palmitoyl-2-(9′-oxo-Nonanoyl)-sn-Glycero-3-Phosphocholine | 870605 |
| 1-Palmitoyl-2-Azelaoyl-sn-Glycero-3-Phosphocholine | 870600 |
| 1-O-Hexadecyl-2-Azelaoyl-sn-Glycero-3-Phosphocholine | 870601 |
| 1-Pamitoyl-2-Myristoyl-sn-Glycero-3-Phosphocholine | 850454 |
| 1-Palmitoyl-2-Arachidonoyl-sn-Glycero-3-Phosphocholine | 850459 |
| 1-Palmitoyl-2-Acetoyl-sn-Glycero-3-Phosphocholine | 880622 |
| 1-Palmitoyl-2-Oleoyl-sn-Glycero-3-Phosphocholine | 850457 |
| L-α-phosphatidylcholine | 840053 |
| L-α-phosphatidylserine | 840032 |
| L-α-phosphatidylethanolamine | 840022 |
| Sphingomyelin | 860062 |
| Ceramides | 860052 |
| D-erythrosphingosine | 860025 |
| Lysophosphatidylethanolamine | 850095 |
| L-α-lysophosphatidylserine | 850092 |
| Phosphatidylinositol-4-phosphate | 840045 |

Mini-Array II

| | |
|---|---|
| 1,2-Dipropionoyl-sn-Glycero-3-Phosphocholine | 850302 |
| 1,2-Dipalmitoyl-sn-Glycero-3-Phosphocholine | 770355 |
| 1,2-Dipalmitoyl-sn-Glycero-3-Phosphobutanol | 860202 |
| 1-Palmitoyl-sn-Glycero-2,3-Cyclic-Phosphate | 857323 |
| 1-Palmitoyl-2-Oleoyl-sn-Glycero-3-Phosphocholine | 850457 |
| 1,2-Dipalmitoyl-3-Trimethylammonium-Propane | 890870 |
| 1,2-Dipalmitoyl Ethylene Glycol | 800604 |
| 1,2-Dipalmitoyl-sn-Glycero-3-Galloyl | 870412 |
| 1-Palmitoyl-2-Oleoyl-sn-Glycero-3-[Phospho-L-Serine] | 840034 |
| 1-Palmitoyl-2-Oleoyl-sn-Glycerol | 800815 |
| 1-Palmitoyl-2-Glutaroyl-sn-Glycero-3-Phosphocholine | 870602 |
| 1-Palmitoyl-2-Oleoyl-sn-Glycero-3-Phospho(TEMPO)choline | 810609 |
| 1-Palmitoyl-2-Oleoyl-sn-Glycero-3-Phosphoethanolamine | 850757 |
| 1-Palmitoyl-2-Oleoyl-sn-Glycero-3-[Phospho-rac-(1-glycerol)] | 840457 |
| 1-Palmitoyl-2-Oleoyl-sn-Glycero-3-Ethylphosphocholine | 890705 |
| 1-Palmitoyl-2-Oleoyl-sn-Glycero-3-Phosphate 840857 | 840857 |

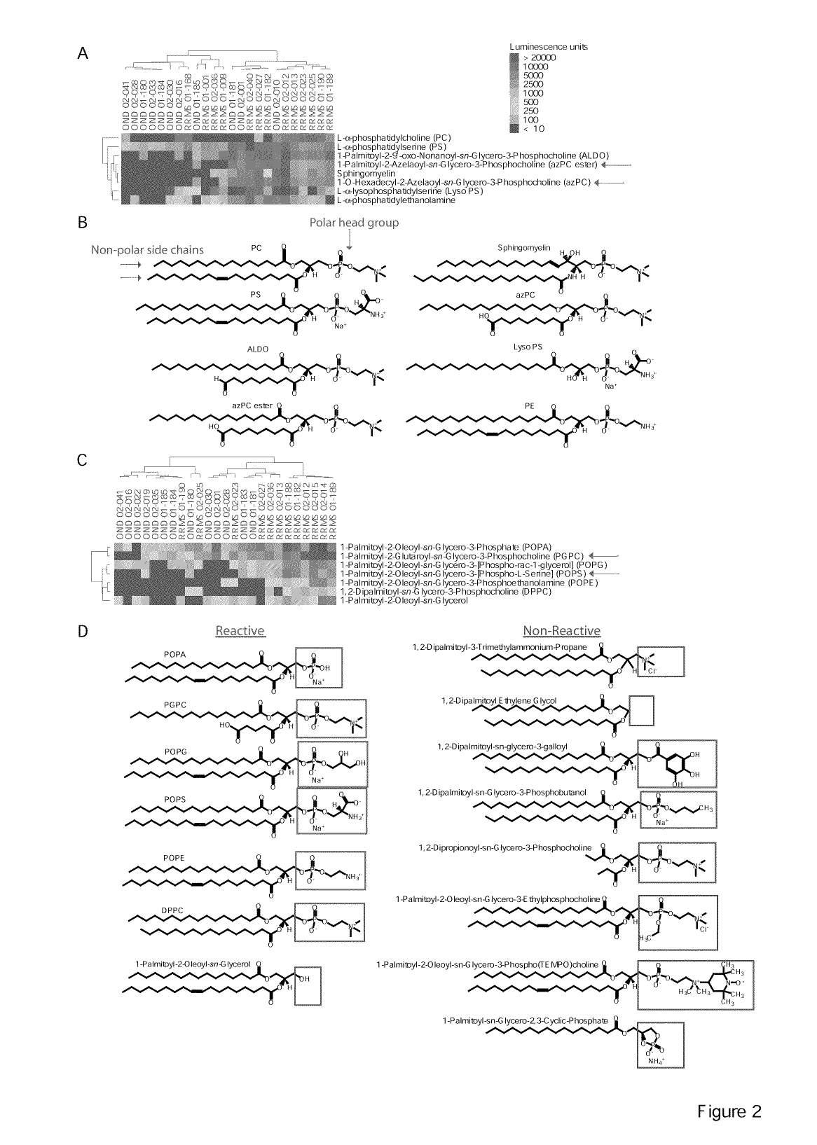

Because we observed significant differences between RRMS and OND samples only when IgG secondary antibody was used, all experiments shown in FIG. 2 were performed with anti-IgG secondary antibody. SAM analysis revealed autoantibody reactivity to 3 of the 7 glycerol-3-phosphocholine-containing lipids (FIG. 2A). All 8 of the lipids identified as targets with the lowest FDR (0.029) had a phosphate group linked to a nitrogen moiety through two carbons (FIG. 2B). The non-polar portion of the targeted lipids contained either one or two side chains, and in some of these lipids, the second side chain had a terminal carboxyl group (FIG. 2B). This suggests that autoantibodies present in RRMS CSF target the phospholipids' phosphate head group, and that the affinity of antibody-lipid binding is not specific to a particular phospholipid. In support of this idea, autoantibodies in MS consistently targeted sphingomyelin (3) (FIGS. 1A, 2A and B) but did not target ceramide, which is sphingomyelin without the phosphate polar head group.

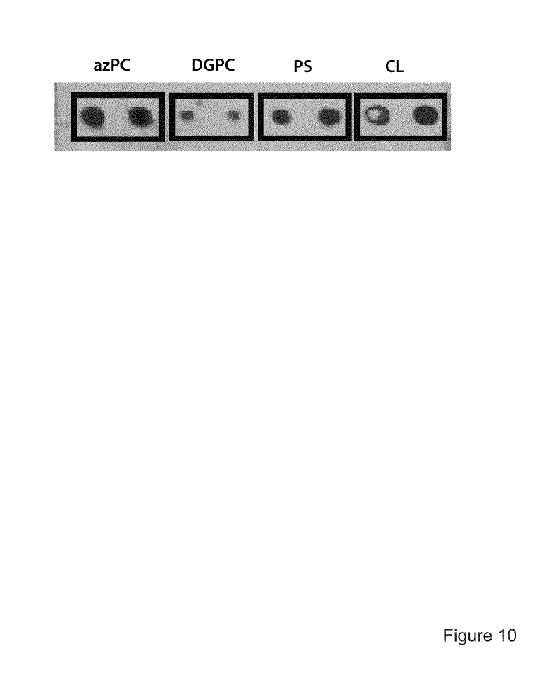

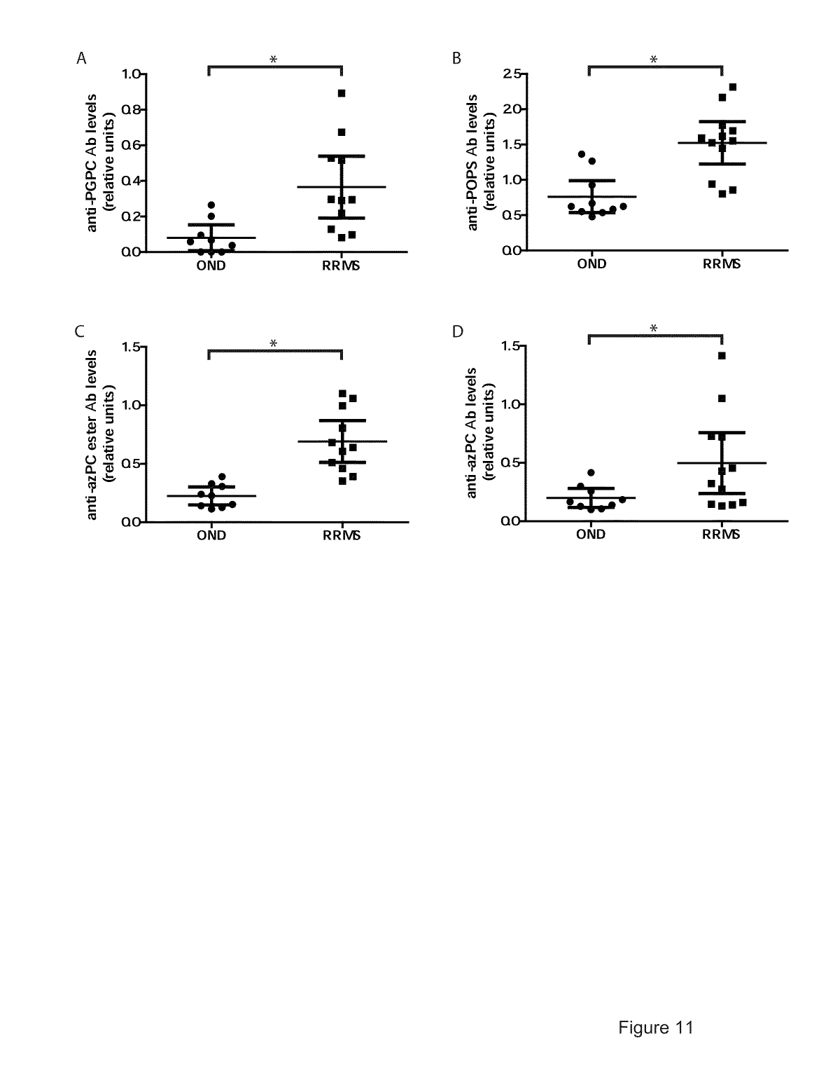

To further investigate the structural basis of the lipids' antigenicity, we examined autoantibody reactivity to an additional 14 lipids that contain various head-group and side-chain modifications of PGPC. The lipids in this Mini Array II are listed in Table 1. We probed Mini Array II with CSF samples from RRMS and OND control patients using a sample set similar to that used in the previous array experiments, and identified autoantibody reactivity to many PGPC-related lipids (FIG. 2C). We noted that 6 of the 7 targeted lipids had a phosphate group, in most cases attached through two carbons to another polar moiety such as nitrogen or oxygen (FIG. 2D). One such lipid, 1-Palmitoyl-2-Oleoyl-sn-Glycerol, contained only a hydroxyl group at this position. Although several of the targeted lipids are endogenously synthesized in human brain, 1,2-dipalmitoyl-sn-glycero-3-phosphocholine (DPPC), a synthetic cationic surfactant that possesses the same head group as PGPC, was also targeted. Of the lipids that were not targeted, 1,2-Dipropionoyl-sn-Glycero-3-Phosphocholine (DGPC, right column, 5^{th} lipid) had a phosphate head group with a structure similar to that of the targeted lipids. Unlike the targeted lipids, however, DGPC did not contain long side chains, suggesting that the lipid side chain may also facilitate antibody binding. (Despite having only short side chains, DGPC bound to the PVDF membrane on our array (FIG. 10)). The other 6 lipids that were not targeted either lacked a phosphate group or contained a phosphate group connected to a bulky group, e.g., a ring structure or a phosphate group linked to 4 carbons (FIGS. 2, C and D). Using ELISA analysis, we confirmed that PGPC, 1-hexadecyl-2-azelaoyl-sn-glycero-3-phosphocholine (azPC), azPC ester, and 1-Palmitoyl-2-Oleoyl-sn-Glycero-3-[Phospho-L-Serine] (POPS) are indeed targeted by autoantibodies, and that the levels of these autoantibodies are significantly higher in CSF from patients with RRMS than in CFS from patients with OND (FIG. 11).

Thus, binding of RRMS CSF autoantibodies to these lipids is dependent on the presence of (i) a non-bulky polar head group such as a phosphate group and (ii) at least one long hydrocarbon side chain.

Antibody Targets are Natural Brain Lipids.

To determine whether the lipids identified as targets of the autoantibody response (FIGS. 1A, 2A,C) are present in MS brain lesions, we performed lipidomic mass spectrometric analysis of pathological specimens taken at autopsy from the brains of MS patients, as previously described. Lipids detected in MS brain lesions included phosphatidylcholines, phosphatidylethanolamines, phosphatidylinositols, phosphatidylglycerols, phosphatidylserines, phosphatidic acids, sphingomyelins, sulfatides, cerebrosides, ceramides, and lysophosphatidylcholines (Table 2).

TABLE 2
Levels of phospholipids in age-matched MS and normal brain samples, as
assessed by shotgun lipidomics. The lipid classes identified are shown.
Total phospholipid levels in control and MS
brain samples (nmol/mg protein)

| | Control 1 | Control 2 | MS 1 | MS 2 |
|---|---|---|---|---|
| Phosphatidylcholine | 219.04 | 186.92 | 172.91 | 150.46 |
| Phosphatidylethanolamine | 380.49 | 380.15 | 318.50 | 310.83 |
| Phosphatidylinositol | 6.85 | 6.94 | 8.77 | 3.38 |
| Diacylglycerol | 1.07 | 1.12 | 0.63 | 0.49 |
| Phosphatidylserine | 169.14 | 157.94 | 122.95 | 61.12 |
| Phosphatidic acid | 2.79 | 2.03 | 2.20 | 1.60 |
| Sphingomyelin | 62.50 | 60.47 | 63.34 | 54.32 |
| Sulfatides | 85.10 | 79.73 | 82.60 | 79.23 |
| Cerebrosides | 291.85 | 312.65 | 556.09 | 213.15 |
| Ceramides | 12.22 | 8.81 | 14.35 | 7.09 |
| Lysophosphatidylcholine | 1.51 | 2.27 | 1.76 | 2.28 |

*MS 1 = relapsing remitting MS; MS 2 = secondary progressive MS

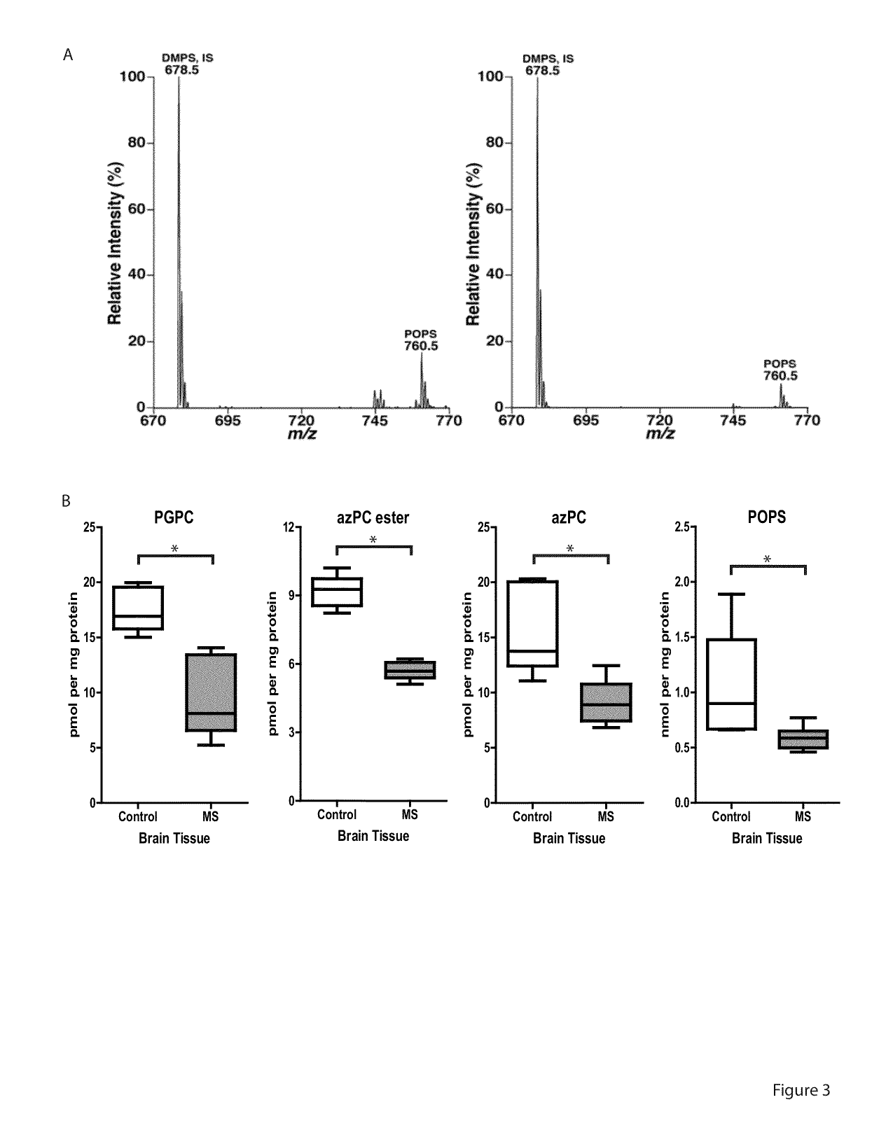

We next asked whether PGPC, azPC, azPC ester, and POPS, specific lipid targets of the autoantibody response in MS (FIGS. 1A, 2 and FIG. 11), are present in MS brain lesions. Mass spectrometric analysis demonstrated the presence of (POPS) at 0.5-1.3 nmol per mg protein (FIG. 3A, B) in MS brain lesions. Because PGPC, azPC, and azPC ester were not detected in our initial mass spectrometric analysis, we used single reaction monitoring (SRM) to test for the presence of these specific lipids in healthy control and MS brain samples. Using SRM, we detected azPC, azPC ester, and PGPC at 5-20 pmol per mg of protein (FIG. 3B). All four lipids were detected both in MS samples and in age-matched control samples, and levels of azPC, azPC ester, PGPC, and POPS were significantly lower in MS samples (FIG. 3B and Table 3), consistent with the observed decrease in phosphatidylcholine levels (Table 2). We confirmed the presence of PGPC, azPC, and azPC ester in human brain extract by liquid chromatography high-resolution mass spectrometry (LC-HRMS), using three criteria: accurate mass agreement with calculated mass, retention time on the column in comparison to authentic standards, and MS/MS fragmentation patterns in comparison to authentic standards.

TABLE 3
Levels of PGPC, azPC ester, azPC, and POPS in MS and age-matched healthy brain samples,
as assessed by single-reaction monitoring.
Content of individual lipid species levels in control and MS brain samples

| | Monoisotopic | | | | | | | | | | | | | per mg | |
|---|---|---|---|---|---|---|---|---|---|---|---|---|---|---|---|
| Species | mass | C1 | C2 | C3 | C4 | C5 | C6 | MS1 | MS2 | MS3 | MS4 | MS5 | MS6 | protein | p value |
| PGPC | 616.38 | 19.98 | 16.03 | 16.52 | 17.33 | 19.42 | 15.02 | 5.23 | 14.06 | 13.21 | 7.46 | 8.76 | 7.01 | pmol | 0.000640 |
| azPC ester | 658.46 | 8.88 | 9.58 | 8.67 | 9.56 | 10.21 | 8.23 | 5.48 | 5.50 | 6.22 | 5.12 | 5.86 | 6.01 | pmol | 0.000001 |
| azPC | 672.44 | 13.30 | 19.96 | 14.22 | 12.85 | 20.31 | 11.06 | 7.62 | 12.45 | 10.22 | 6.82 | 5.31 | 8.49 | pmol | 0.006488 |
| POPS | 750.51 | 0.67 | 1.89 | 0.88 | 0.92 | 1.34 | 0.66 | 0.61 | 0.51 | 0.77 | 0.46 | 0.59 | 0.58 | pmol | 0.038233 |

MS 1, MS 5, & MS 6 = relapsing remitting MS;
MS 2 = secondary progressive MS;
MS 3 & MS 4 = chronic MS

Antibody-Targeted Oxidized Lipids Treat Established EAE.

To investigate the therapeutic potential of oxidized phosphatidylcholine derivatives of PGPC in EAE, we administered the initial dose of POPS, PGPC, azPC, or azPC ester to mice with established EAE. We administered the lipids systemically to establish a delivery modality appropriate for future translational studies in humans. The first dose of 100 μg of lipid was injected intravenously into the tail of mice once they developed tail or hind-limb paralysis, and the lipid treatment was repeated every other day, such that 10 injections were administered intravenously during the course of EAE. POPS, PGPC, azPC, and azPC ester were all able to ameliorate established EAE (FIG. 4A).

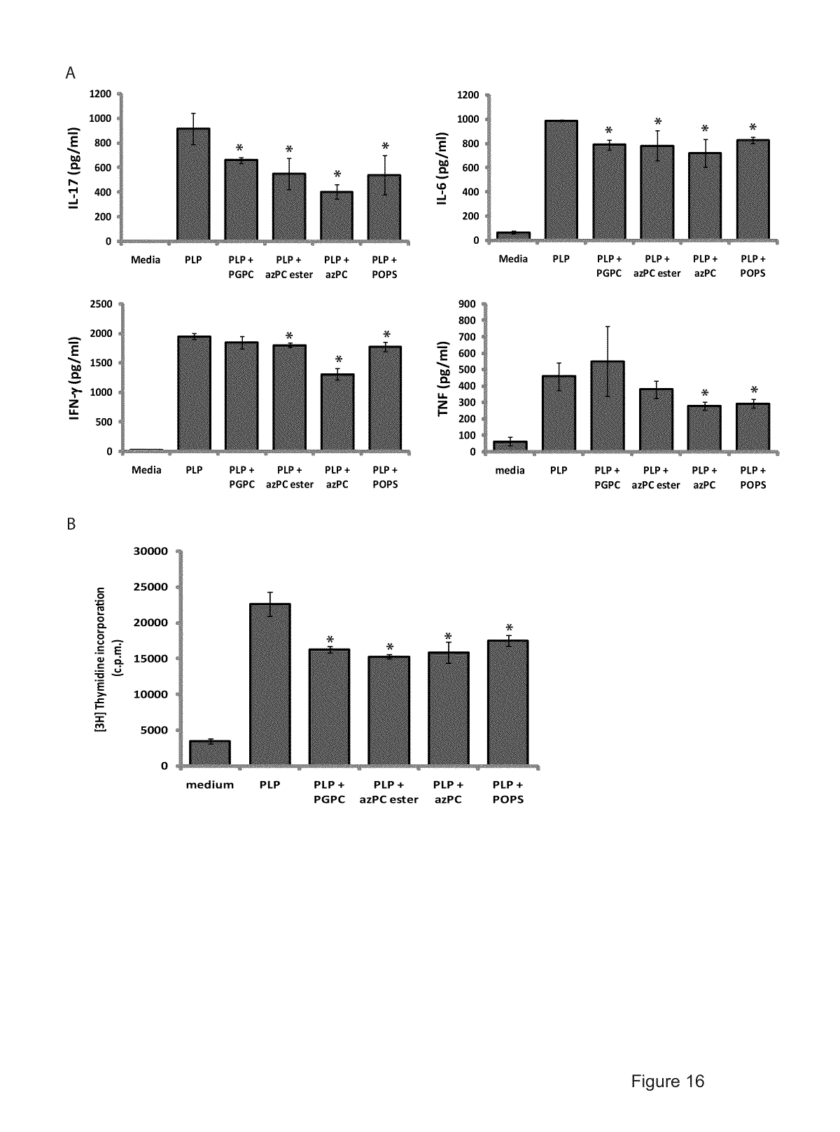

Antibody-targeted lipids suppress autoreactive T cells in vitro. To investigate the mechanisms by which oxidized phosphatidylcholine derivatives and POPS provide therapeutic benefit in EAE (FIGS. 1B and 4A), we assessed the ability of the lipids to directly inhibit the T-cell-mediated inflammatory responses that underpin EAE. We assessed the effect of these lipids on MBP_{Ac1-11}-induced cytokine production by naïve splenocytes from mice transgenic for the myelin basic protein (MBP)_{Ac1-11}-specific T-cell receptor. azPC reduced MBP_{Ac11-11}-induced T-cell production of the inflammatory cytokines IFN-γ, TNF, and IL-6. POPS also reduced TNF and IL-6 levels and but did not significantly reduce IFN-γ levels. PGPC and azPC ester reduced levels of some of the cytokines, but these effects were less dramatic than the effects of POPS and azPC (FIG. 4B). Neither POPS nor the oxidized phosphatidylcholine derivatives affected production of IL-12p40. These lipids had similar inhibitory effects on PLP₁₃₉₋₁₅₁-induced IL-17, IL-6, IFN-γ and TNF production by splenocytes from PLP₁₃₉₋₁₅₁-immunized mice (FIG. 16A).

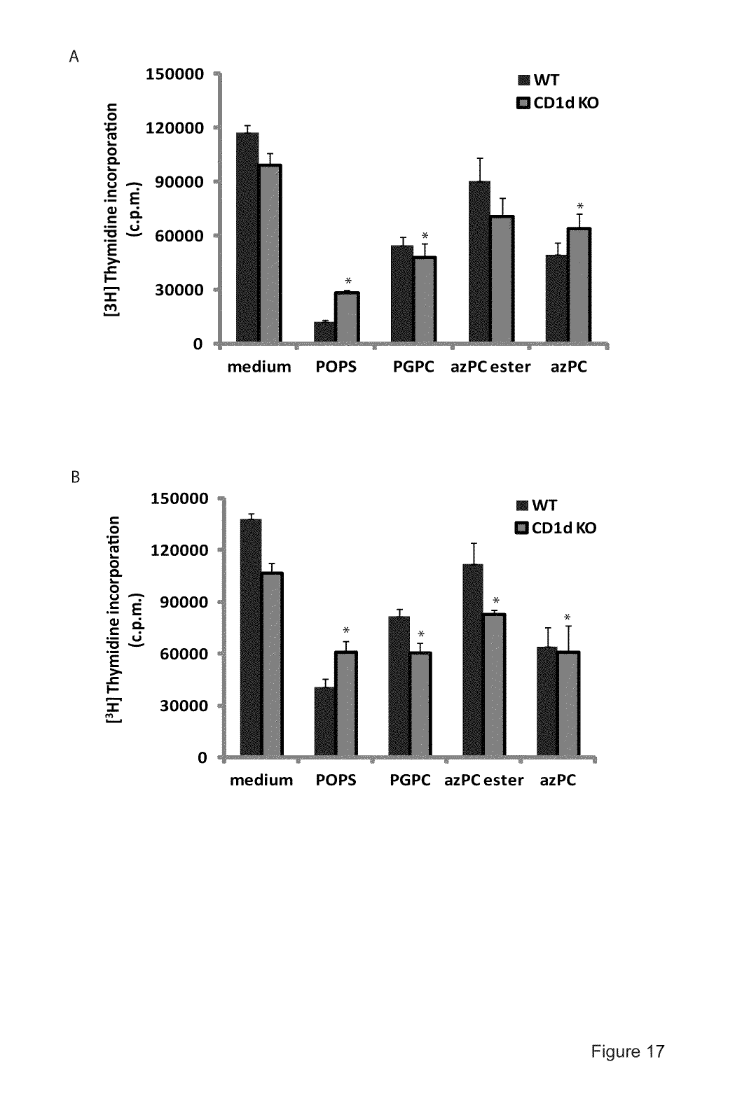

To determine whether these lipids affect T-cell proliferation, we measured ³H-thymidine incorporation in splenocytes stimulated with MBP_{Ac1-11} in the presence of the different lipids. Sulfatide which worsened EAE and sphingomyelin which did not treat established EAE, were used as controls in these assays. POPS, PGPC, azPC, and azPC ester reduced T-cell proliferation in response to MBP_{Ac1-11}, whereas sulfatide and sphingomyelin did not (FIG. 4C). We obtained similar results with PLP₁₃₉₋₁₅₁-stimulated splenocytes from mice immunized with PLP₁₃₉₋₁₅₁ (FIG. 16B), as well as with T cells stimulated with anti-CD3 and anti-CD28 antibodies (white bars, FIG. 17). Studies using T cells from mice deficient in CD1 showed that the lipid-mediated suppression of T-cell responses occurred through a CD1-independent mechanism (FIG. 17).

azPC, azPC-Ester, PGPC and POPS Induce Apoptosis of Immune Cells.

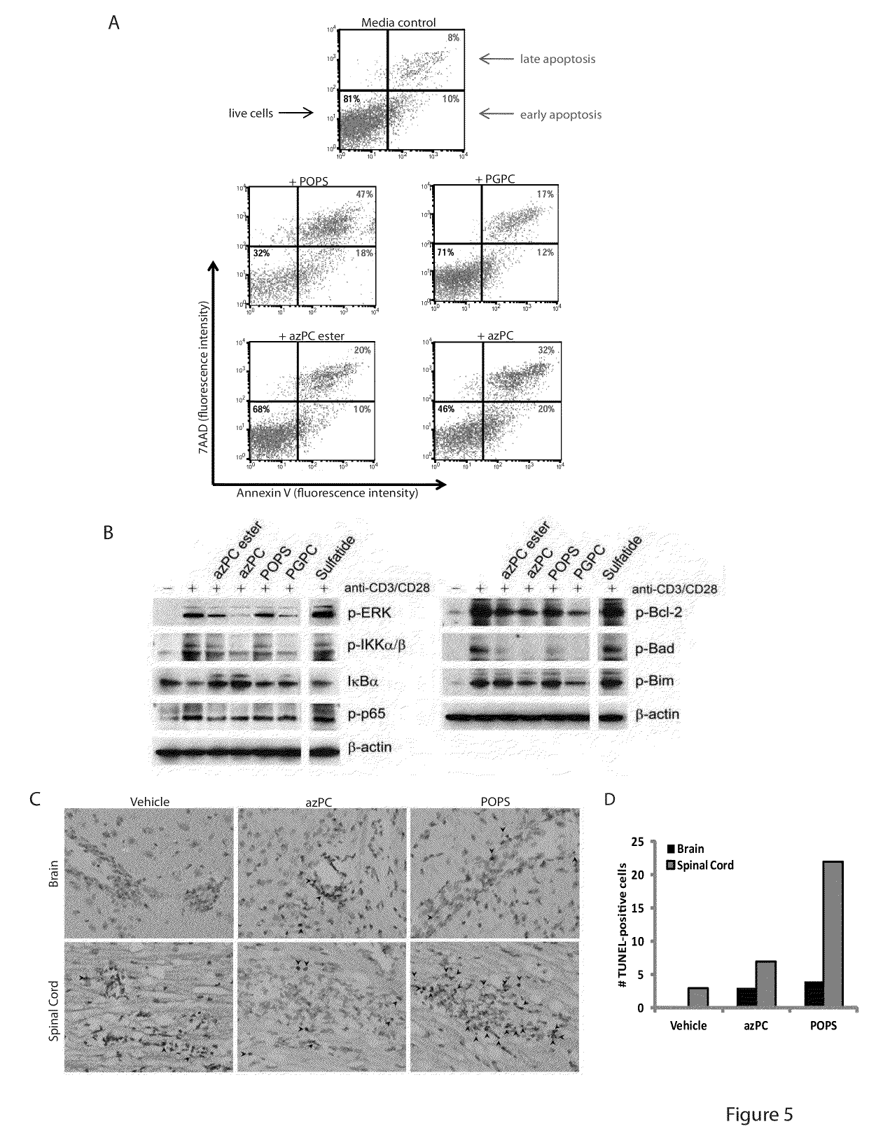

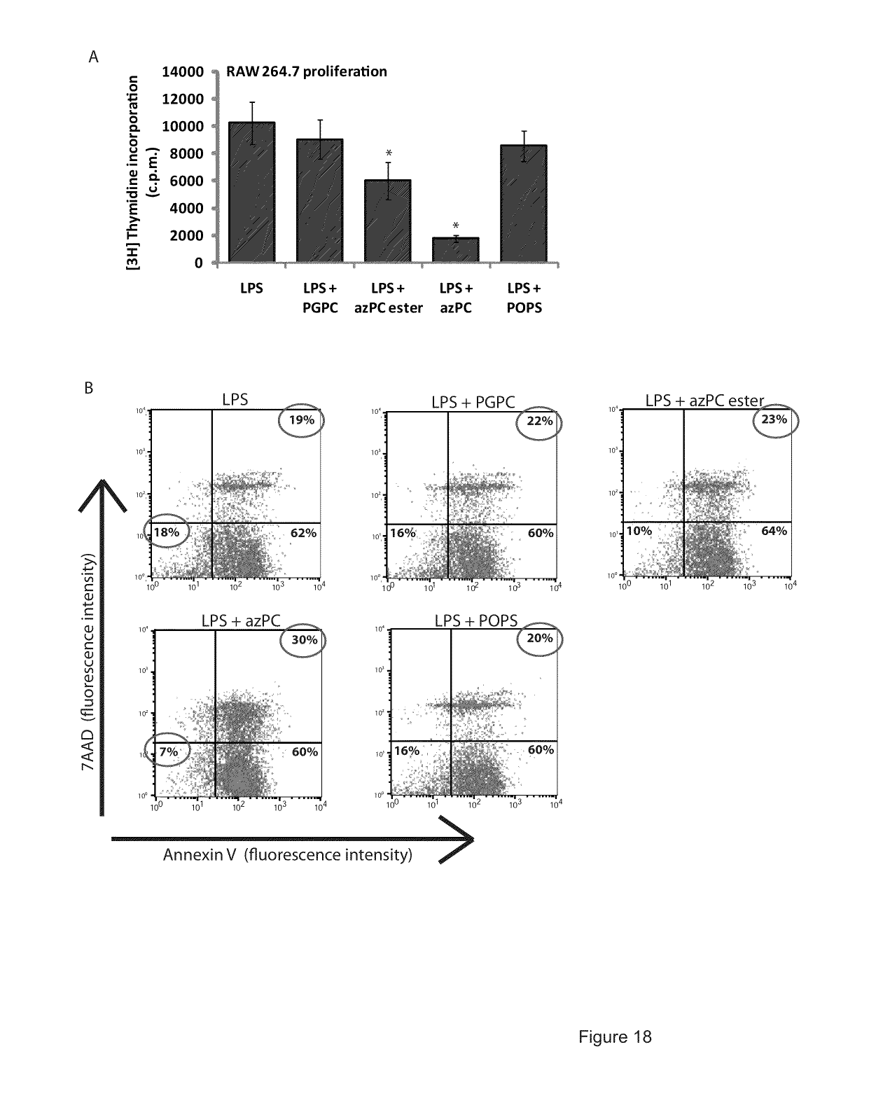

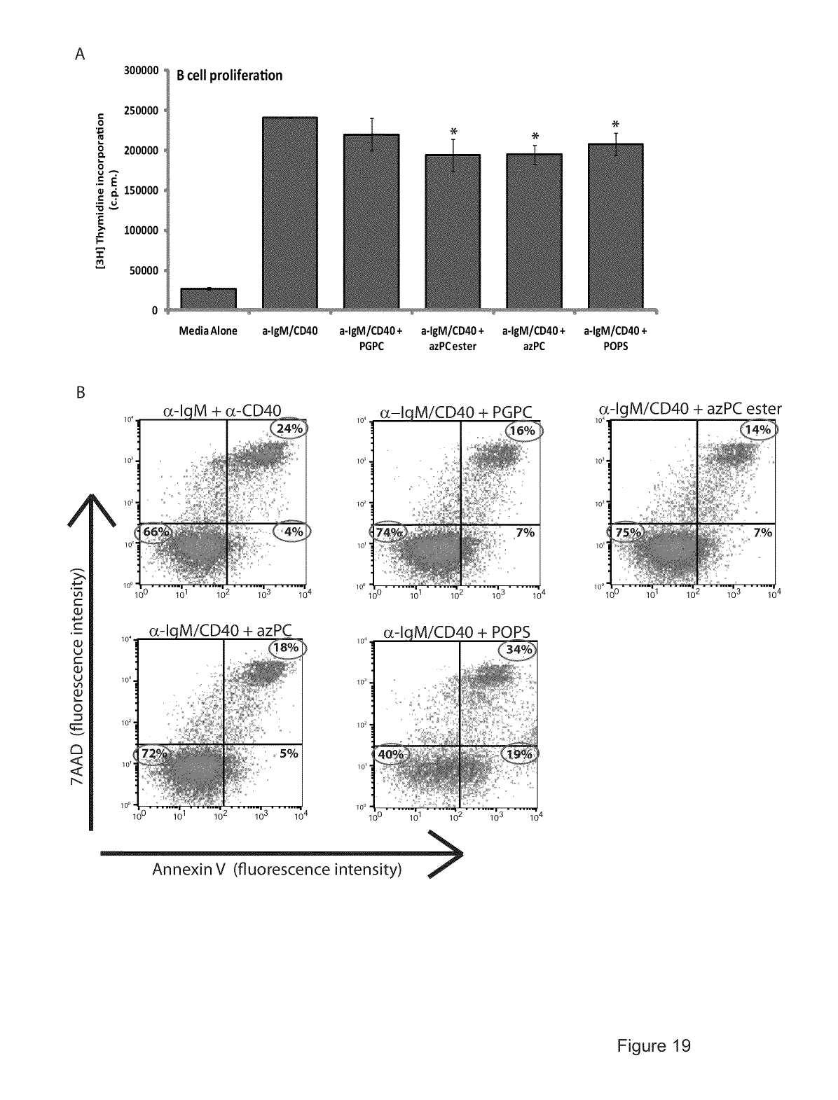

To assess whether programmed cell death contributed to the lipid-mediated reduction in proliferation of activated T cells, we performed Annexin V staining and 7-amino-actinomycin D (7AAD) uptake assays, which enable identification of early apoptotic (AnnexinV+, 7AAD−) and late apoptotic (AnnexinV+, 7AAD+) cells (FIG. 5A). At 48 hours, azPC ester or PGPC increased T-cell apoptosis 2-3-fold, azPC increased apoptosis >4-fold, and POPS >5-fold. The lipids had differential effects on other cell types, however. azPC and azPC ester suppressed proliferation (FIG. 18A), while only azPC increased apoptosis (FIG. 18B) of an LPS-activated mouse macrophage cell line (RAW 264.7). In contrast, azPC, azPC ester, and POPS modestly suppressed proliferation of purified anti-IgM F(ab′)2 fragment/anti-CD40-activated mouse B cells (FIG. 19A), and only POPS increased apoptosis of the B cells (FIG. 19B).

To determine whether lipid treatment induced apoptosis in vivo in the context of EAE, we performed TUNEL staining on brains and spinal cord sections from EAE mice treated with azPC or POPS. POPS and azPC increased the number of TUNEL-positive cells with lymphocyte morphology in the brain and spinal cord of mice with EAE (FIG. 5C,D).

azPC, azPC-Ester, PGPC and POPS Induce Pro-Apoptotic and Proinflammatory Pathways.

To investigate the molecular mechanisms underlying the lipid-mediated modulation of T cells, we examined the effects of these lipids on inflammatory, survival, and apoptotic signaling pathways. The transcription factor nuclear factor-kappaB (NF-κB) plays a crucial role in the activation, survival, and proliferation of T cells by driving the transcription of proinflammatory cytokine genes (including IFN-γ, TNF, and IL-6), anti-apoptotic genes, and genes involved in cell cycle progression. Deficiency in NF-κB signaling suppresses the expansion of autoreactive T cells. Likewise, extracellular signal-regulated kinase (ERK) activity is integral to cell proliferation and survival. Costimulation of T cells with anti-CD3 and anti-CD28 antibodies activated the canonical NF-κB pathway, as indicated by an increase in phosphorylation of the activating kinases IKKα and IKKβ; a decrease in levels of the NF-κB inhibitor IκBα; and an increase in serine 536-phosphorylation of p65, the major transactivating subunit of NF-κB (FIG. 5B). ERK activity was also induced. POPS, PGPC, azPC, and azPC ester each suppressed the CD3/CD28-induced activation of the NF-κB and ERK pathways (FIG. 5B). These lipids also suppressed the CD3/CD28-induced activity (phosphorylation) of B-cell lymphoma protein-2 (Bcl-2) (FIG. 5B), an important anti-apoptotic protein. Bcl-2-interacting molecule (Bim) and Bad, pro-apoptotic members of the Bcl-2 family, antagonize the anti-apoptotic activity of Bcl-2. Importantly, Bim plays critical roles in both activation and apoptosis of autoreactive T cells in EAE. ERK-mediated phosphorylation of Bim at serine 69 suppresses the apoptotic activity of Bim, while the NF-κB pathway suppresses Bim expression. Similarly, ERK-mediated phosphorylation of Bad at serine 112 inhibits the apoptotic activity of Bad. Collectively, POPS, PGPC, azPC, and azPC ester suppressed the CD3/CD28-induced phosphorylation of Bim and Bad at serines 69 and 112, respectively. Sulfatide, which worsens EAE, neither inhibited the NF-κB and ERK pathways, nor suppressed the phosphorylation of the apoptotic Bcl-2 family proteins (FIG. 5B).

Together, these results demonstrate that suppression of the NF-κB and ERK pathways, and the resulting derepression of apoptotic pathways, underlies the anti-inflammatory and anti-proliferative effects of the oxidized phosphatidylcholine derivatives and POPS.

Saturated Fatty-Acid Side Chains Mediate T-Cell Suppression.

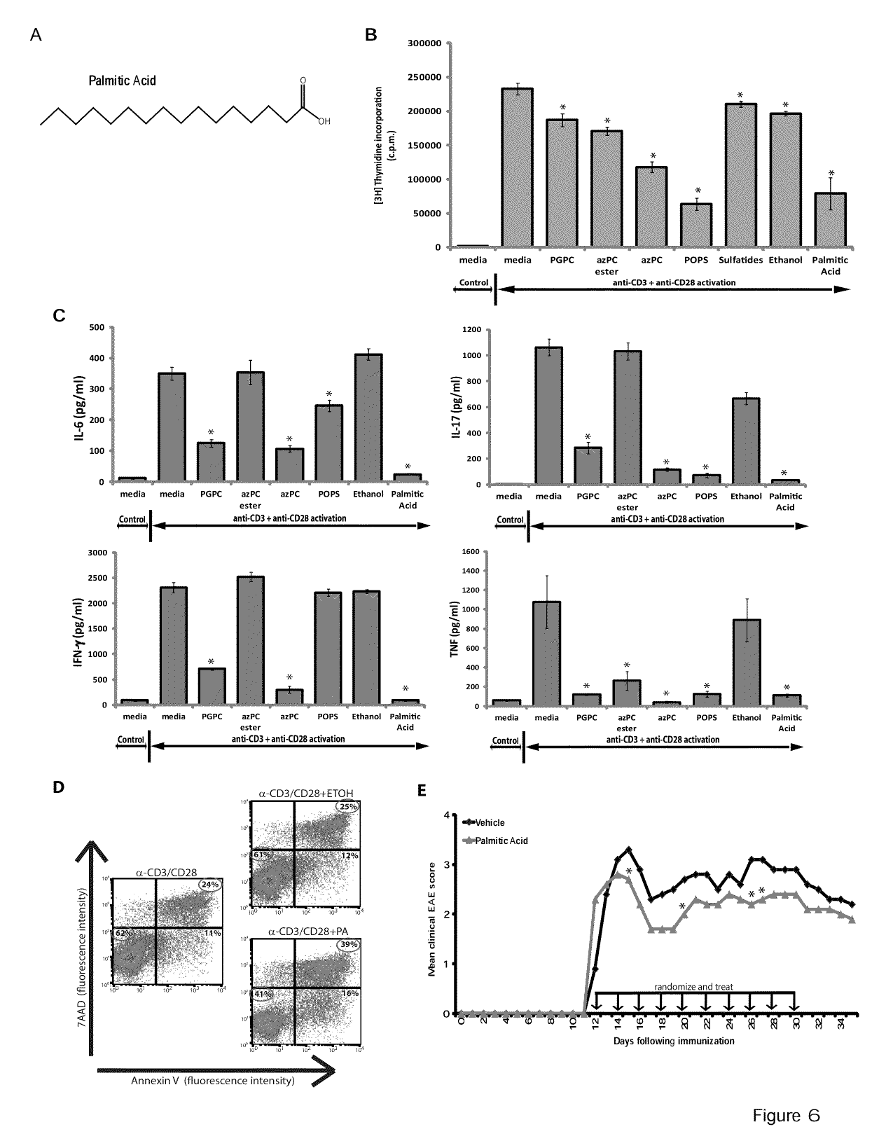

Unsaturated fatty-acid-derived mediators generated in resolving exudates can contribute to the termination of an inflammatory response. We asked whether the saturated fatty-acid side chains within POPS, PGPC, azPC, and azPC ester could modulate T-cell proliferation. Fatty acids esterified to the phosphate head group at the sn-1 position, and the oxidizable fatty acids at the sn-2 position, were of particular interest in light of their potential roles in modulating inflammation. We tested a variety of structural analogs of these fatty-acid side chains, including palmitic acid, ethyl palmitate, sebacic acid, octanoic acid, methyl octanoate, and suberic acid. These molecules form part of the saturated, non-polar side chain of our lipids of interest, which may be cleaved from the phospholipids by lipases that are upregulated in MS brain. Palmitic acid (FIG. 6A) suppressed T-cell proliferation at a concentration of 0.1 mM, while sebacic acid, octanoic acid, and methyl octanoate suppressed proliferation at 1.0 mM, 5 mM, and 10 mM, respectively.

We next compared the effect of the lipids or palmitic acid on anti-CD3- and anti-CD28-stimulated purified naïve T cells (FIG. 6B). Palmitic acid suppressed T-cell proliferation as effectively as the phospholipids. It also suppressed T-cell production of the inflammatory cytokines IL-6, IL-17, IFN-γ and TNF (FIG. 6C), and induced T-cell apoptosis (FIG. 6D).

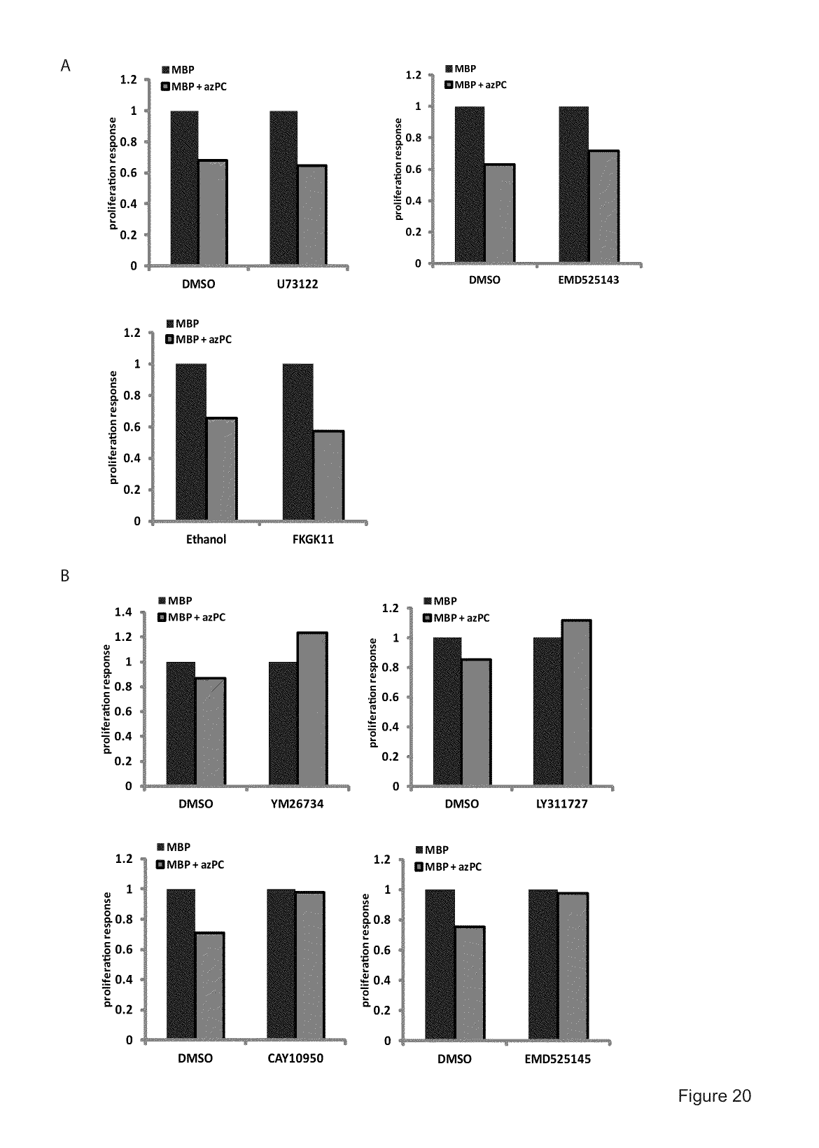

In vivo, administering palmitic acid at the time of disease onset attenuated EAE, and disease relapsed once the palmitic acid treatment was halted (FIG. 6E), indicating that this fatty-acid side chain is itself therapeutically efficacious. Further, we identified palmitic acid as a fatty-acid side chain present in both oxidized lipids as well as in its free form in both MS and healthy brain (Table 4). We also tested the effect of inhibiting specific phospholipases including PLC (with inhibitor U73122), PLA₂ (OBAA), cPLA_{2α} (EMD525143), iPLA₂ (FKGK11), sPLA₂ Groups IIA, IID, IIE, V, X (YM 26734), sPLA₂ Groups IIA, V (LY 311727), sPLA₂ Group V (CAY10590), or sPLA₂ Group IIA (EMD525145), for their effects on azPC-mediated inhibition of MBP-specific T-cell proliferation. We found that pre-incubating T cells with all four sPLA₂ inhibitors suppressed the effects of azPC (i.e. MBP-stimulated T-cell proliferation was partially restored), whereas pre-incubating the T cells with various concentrations of the other phospholipase inhibitors had no effect or simply killed the T cells (FIG. 20 and Table 5). Together, these results suggest that the non-esterified fatty-acid side chains of the targeted lipids are responsible for their T-cell suppressive properties.

TABLE 4
Total content of palmitic acid in each individual phospholipid
class in age-matched MS and normal brain samples, as
assessed by single reaction monitoring.
Total content of palmitic, acid in each individual
phospholipid class (nmol/mg protein)

| | Control 1 | Control 2 | MS 1 | MS 2 |
|---|---|---|---|---|
| in Phosphatidylethanolamines | 32.69 | 27.57 | 24.97 | 23.92 |
| in Phosphatidylcholines | 108.17 | 110.27 | 109.11 | 98.38 |
| in Phosphatidylserines | 0.38 | 0.17 | 0.56 | 0.27 |
| in Phosphatidylinositols | 0.74 | 0.83 | 1.24 | 0.26 |
| in Phophatidylglycerols | 0.36 | 0.34 | 0.26 | 0.13 |
| in Phosphatidic acids | 0.37 | 0.30 | 0.26 | 0.39 |
| in Lysophosphatidylcholines | 0.50 | 0.67 | 0.58 | 0.70 |
| unbound | 4.75 | 7.81 | 6.65 | 4.60 |
| Total | 147.96 | 147.96 | 143.63 | 128.65 |

* MS 1 = relapsing remitting MS; MS 2 = secondary progressive MS

TABLE 5
Phospholipase inhibitor effect on azPC-mediated inhibition
of MBP Ac1-11 specific T cell proliferation.
Effects of phospholipase inhibitors on azPC-mediated
inhibition of MBP-specific T cell proliferation

| Inhibitors | Target | Effect on azPC |
|---|---|---|
| U 73122 | PLC | No effect |
| Tocris | | |
| 525143 | cPLA_{2α} | No effect |
| EMD | | |
| FKGK 11 | iPLA₂ | No effect |
| Cayman | | |
| YM 26734 | sPLA₂ | Inhibited |
| Tocris | Groups IIA, IID, IIE, V, X | (decreased potency) |
| LY 311727 | sPLA₂ | Inhibited |
| Tocris | Groups IIA, V | (decreased potency) |
| CAY10950 | sPLA₂ | Inhibited |
| Cayman | Group V | (decreased potency) |
| 525145 | sPLA₂ | Inhibited |
| EMD | Group IIA | (decreased potency) |

We report the use of two lipidomic technologies—lipid antigen microarrays and lipidomic mass spectrometry—to identify myelin lipids targeted by autoantibody responses in MS. Furthermore, we show that these autoantibody-targeted phospholipids ameliorate disease in a mouse model of MS by inhibiting the autoaggressive T-cell responses that underpin autoimmune demyelination. Oxidized phosphatidylcholine derivatives inhibited MBP-specific T-cell proliferation and cytokine production, and induced the apoptosis of these autoaggressive cells.

At the molecular level, the anti-inflammatory effects of the lipids were associated with the inhibition of NF-κB and ERK, signaling molecules that promote inflammation and cell survival, and the derepression of Bim and Bad, the molecular executors of programmed cell death. Our observation that POPS induces T-cell apoptosis is confirmed by our in vivo results demonstrating increased apoptosis of lymphocytes in the perivascular cuffs of brain and spinal cord lesions of azPC- and POPS-treated mice with EAE. Intriguingly, osteopontin, a protein that induces relapse and progression of EAE, has the opposite effect: it promotes the survival of MBP-reactive T cells by activating the NF-κB pathway and inhibiting Bim-mediated apoptosis. Indeed, T-cell apoptosis is a key mechanism by which autoimmune attack on the CNS is kept in check, and it plays an important role in spontaneous EAE remission.

In MS brain lesions, inflammation leads to an increase in nitric oxide, which can oxidize lipid components of the brain. Using mass spectrometry, we demonstrated the presence of oxidized phospholipids—and specifically of the autoantibody-targeted therapeutic lipids—in normal and in MS brain. Levels of oxidized phosphatidylcholine derivatives were lower in MS brain than in healthy brain. This may reflect the autoimmune-mediated destruction and elimination of myelin sheath lipids that occurs in MS, such that levels of oxidized phospholipids are diminished despite an increase in lipid peroxidation. Antibodies against oxidized phosphatidylcholine are deposited in MS lesions, and such autoantibody targeting of lipids could contribute to MS pathogenesis by reducing the levels or blocking the activity of the protective lipids. By binding to the oxidized phosphocholines and phosphoserines, the anti-lipid autoantibodies could promote MS pathogenesis in a variety of ways: they could damage the myelin sheath; inhibit the pro-apoptotic or immunoregulatory function of these lipids; and/or enhance clearance of these anti-inflammatory lipids. Further, our findings that that the amounts of oxidized lipids are lower in MS brain lesions, that their fatty-acid side chains are anti-inflammatory, and that their polar heads targeted by autoantibodies suggest that autoantibodies targeting the polar head groups of PGPC and its derivatives could reduce their anti-inflammatory effects on T cells by depleting these anti-inflammatory lipids or by abrogating the generation of protective free fatty acids from these lipids.

Oxidized phospholipids are conventionally considered proinflammatory and may exacerbate inflammation-associated disease. Whereas oxidized phosphatidylcholine was previously identified as a marker of neuroinflammation in MS, our findings suggest that derivatives of oxidized phosphatidylcholine in fact function as part of an endogenous feedback mechanism that attenuates adaptive autoimmune responses in the brain. Indeed, oxidized phospholipids are emerging as Janus-like molecules: whereas their pathogenic role in atherosclerosis is well established, these lipids play a protective, anti-inflammatory role in endotoxin-induced tissue damage by inhibiting Toll-like receptor-4 signaling. Thus, the role of oxidized phospholipids in physiology and pathophysiology is context dependent. In brain, derivatives of oxidized phospholipids are important mitigators of autoimmune responses.

The saturated, non-polar side chains mediate the protective effects of the therapeutic lipids. Palmitic acid, representative of such fatty-acid side chains present within the lipids, was able to reproduce the therapeutic effects of the lipids in vitro and in vivo. Regardless of whether they act as free fatty acids or as phospholipid components, these saturated fatty acids appear to serve a function similar to that of the unsaturated fatty-acid derivatives resolvins and protectins, which mediate resolution of inflammation in tissue exudates.

The use of lipidomics for drug discovery provides unique opportunities. Our results show that the immune system may drive autoimmune disease by abrogating the protective effects of molecules involved in inflammatory homeostasis. Choosing the target of the antibody as a potential therapeutic, in this case the lipids identified on an autoantibody array, provides a fresh strategy for screening therapeutics. We found that the phosphocholine head group is an important determinant of the antigenicity of brain lipids, a discovery that enabled the identification of additional therapeutic lipids through stringent statistical analysis of lipid microarray data combined with lipidomic mass spectrometric analysis. The identification of lipids targeted by autoantibodies affords the opportunity to mine small lipid-soluble molecules as potential new drugs for autoimmune disease.

### Materials and Methods

Reagents. We obtained POPS, PGPC, sphingomyelin, sulfatide, azPC, azPC ester, and all other phosphatidylcholine derivatives listed in FIG. 8 from Avanti Polar Lipids. Palmitic acid was purchased from Sigma. Proteolipid protein (PLP)₁₃₉₋₁₅₁ (HCLGKWLGHPDKF) and MBP_{Ac1-11} (ASQKRPSQRHG) were synthesized and HPLC-purified (>97%) by the Stanford PAN facility.

Patient CSF Samples.

All human samples were collected and used under protocols approved by the Institutional Review Boards of the Karolinska Institute and Stanford University. Patient characteristics are listed in Table 1.

Lipid Array Analysis.

Lipid arrays were generated and analyzed as previously described. Briefly, we used a Camag Automatic TLC Sampler 4 robot to print 10 to 100 pmol of lipids on PVDF membranes affixed to the surface of microscope slides. These lipid arrays were probed with 1:20 dilutions of human CSF, followed by 1:8000 dilutions of either anti-human IgG+IgM or anti-human IgG (Jackson Immunoresearch) conjugated to horseradish peroxidase (HRP). Bound HRP-conjugated antibodies were visualized by chemiluminescence (ECL Plus, Amersham) and autoradiography. We used GenePix Pro 5.0 software (Molecular Devices) to extract the net median pixel intensities for individual features from digital images of the array autoradiographs. We applied the SAM algorithm (version 1.21) to identify lipids with statistically significant differences in array reactivity between groups of humans or mice. The list of ‘significant lipids’ with the lowest q value (false discovery rate, FDR) is reported in each heatmap Figure. We arranged the SAM results into relationships by using Cluster software and displayed the results by using TreeView software.

Lipidomic Analysis of Brain Samples.

Archived postmortem samples from MS brain and age-matched healthy brain were analyzed by shotgun lipidomics as previously described. The six MS brain samples analysed were as follows: MS 1, an active lesion from a 59-year-old female with relapsing-remitting MS; MS 2, an active lesion from a 72-year-old male with secondary progressive MS; MS 3, a chronic active lesion from a 47-year-old female with chronic MS; MS 4, a chronic active lesion from a 76-year-old male with chronic MS; MS 5, an acute active lesion from a 31-year-old female in the relapsing phase of relapsing-remitting MS; MS 6, a chronic active lesion from the same 31-year-old female with relapsing-remitting MS. Control brain samples were thoroughly examined to rule out the presence of neurological disease. Control samples were obtained from normal-appearing white matter from the brains of the following individuals: C1, 23-year-old male; C2, 52-year-old female; C3, 23-year-old male; C4, 52-year-old male; C5, 82-year-old male; and C6, 44-year-old female. Samples of healthy brain and samples of MS lesions were pulverized in liquid nitrogen. Lipid extracts were generated and analyzed by electrospray ionization mass spectrometry (typically within 1 week) using a TSQ Quantum Ultra Plus triple-quadrupole mass spectrometer (Thermo Fisher Scientific) equipped with an automated nanospray apparatus (Nanomate HD, Advion Bioscience Ltd.) and Xcalibur system software.

EAE Induction.

Animal experiments were approved by, and performed in compliance with, the National Institute of Health guidelines of the Institutional Animal Care and Use Committee at Stanford University. For induction of EAE, 8- to 12-week-old female SJL/J mice (Jackson Laboratory) were immunized subcutaneously with 100 μg of PLP₁₃₉₋₁₅₁ emulsified in CFA (Difco Laboratories).

Lipid Co-Immunization:

Three injections of PGPC (6 μg/mouse/injection) or vehicle (0.05% Tween-20 in PBS) were delivered on days 0, 4, and 7 after immunization with PLP₁₃₉₋₁₅₁. On day 0, the lipid or vehicle was emulsified together with PLP₁₃₉₋₁₅₁ in CFA and administered by subcutaneous injection. At subsequent time points, lipid or vehicle was injected intraperitoneally. EAE was assessed as previously described.

Lipid and palmitic-acid treatment: Administration of lipid, palmitic acid, or vehicle was initiated once the PLP₁₃₉₋₁₅₁-immunized mice developed paralysis (representing clinical EAE) and repeated every other day, for a total of ten separate injections. 100 μg of POPS, PGPC, azPC ester, azPC, palmitic acid, or vehicle (0.05% Tween-20 in PBS) was administered in 0.2 ml intravenously in the tail.

Proliferation and Cytokine Assays.

Splenocytes were harvested from mice transgenic for the MBP_{Ac1-11}-specific T-cell receptor and stimulated with 2 μg/ml of MBP_{Ac1-11} in the presence of 30 μg/ml of lipid. Lymph nodes and spleens were also harvested from naive C57BL/6 mice, and CD3⁺ T-cell enrichment columns (R&D systems) were used to isolate CD3⁺ T cells. The purified CD3⁺ T cells were stimulated with 5 μg/ml of plate-bound anti-CD3 antibodies and anti-CD28 antibodies in the presence of 30 μg/ml of POPS, PGPC, azPC ester, brain sulfatides, or azPC, 0.25 mM palmitic acid, or 100% ethanol (as the vehicle control). For assessment of proliferation, 1 μCi of ³H-thymidine was added to each well for the final 18-24 hours of culture, and incorporation of radioactivity was measured by using a Betaplate scintillation counter. Cytokine assays were performed on culture supernatants after 24 (IL-12p40) or 48 hours (IFN-γ, IL-6, and TNF) of culture by using the BD OptEIA™ Mouse ELISA kits (BD Biosciences).

Flow Cytometry.

Cells were stained according to standard protocols, run on a FACScan flow cytometer (BD Biosciences), and analysed with CellQuest software (BD Immunocytometry Systems). The antibody conjugate used was FITC anti-CD4, clone GK1.5 (BD Pharmingen). 7AAD staining was performed by using the Annexin V-PE Apoptosis Detection Kit I (BD Pharmingen).

Western Blotting.

CD3⁺ T cells were isolated from the lymph nodes of naïve C57BL/6 mice by using CD3⁺ T-cell enrichment columns (R&D systems). T cells were pre-incubated with 30 μg/ml of lipid for 1 hour at 37° C. degrees and then stimulated with plate-bound anti-CD3 and anti-CD28 antibodies (5 μg/ml, eBiosciences) in the presence of the lipids for 15 minutes or 24 hours. Cells were washed with ice-cold PBS and lysed in RIPA lysis buffer containing 1X Halt protease and phosphatase inhibitor cocktail (Pierce) with a Dounce homogenizer. Immunoblotting was performed with antibodies against phospho-Bim (serine 69), phospho-Bad (serine 112), phospho-Bcl-2 (serine 70), phospho-IKKα/β (serines 180/181), phospho-p65 (serine 536), phospho-ERK1/2 (threonine 202/tyrosine 204), and IκBα from Cell Signaling Technology.

TUNEL Assay.

Mice with EAE were treated intravenously with 200 μg of azPC, 200 μg of POPS, or vehicle (0.05% Tween-20 in PBS) on day 15 after immunization with CFA and PLP₁₃₉₋₁₅₁ peptide. Mice were treated with lipids for 12, 24, or 48 hours and then sacrificed and perfused with 4% paraformaldehyde. Brains and spinal cords were embedded in paraffin and sectioned. TUNEL-positive cells were detected by using the In Situ Cell Death Kit, AP (Roche) according to the manufacturer's instructions.

ELISA for measuring levels of antibodies to POPS, PGPC, azPC, and azPC ester. Lipids dissolved in methanol were added to Corning Costar 3590 enzyme immunoassay plates at 5 nmol/well. The methanol solvent was then evaporated under nitrogen gas. Plates were blocked with BD OptEIA diluent for 3 hours. Standard curves were generated using standardized human serum containing autoantibody against cardiolipin (ImmunoVision). Detection of primary antibodies was achieved using HRP-conjugated goat anti-human IgG antibody (1:10,000).

Luxol Fast Blue Stain.

Membranes were blocked in 1% fat-free bovine serum albumin overnight, washed in 100% ethanol and 95% ethanol, and then incubated in luxol fast blue solution (NovaUltra Stain Kit) at 56° C. overnight. Membranes were immersed in lithium-carbonate solution and then in 70% ethanol, and finally washed with distilled water.

EAE Induction.

To induce EAE in SJL/J mice (Jackson Mice), we immunized 8- to 12-week-old female animals subcutaneously with 100 μg of PLP₁₃₉₋₁₅₁ emulsified in CFA (Difco Laboratories).

Prophylactic administration of lipid: Three injections of PGPC or sphingomyelin (6 μg/mouse/injection) or vehicle (0.05% Tween-20 in PBS) were delivered on days 0, 4, and 7 after immunization with PLP₁₃₉₋₁₅₁. On day 0 the lipid or vehicle was emulsified together with PLP₁₃₉₋₁₅₁ in CFA and administered by subcutaneous injection. For subsequent time points, lipid or vehicle was injected intraperitoneally as previously described. Clinical disease was monitored daily using the following scoring system: 0, no disease; 1, limp tail; 2, hind limb weakness; 3, hind limb paralysis; 4, hind limb and forelimb paralysis; 5, death. Animal experiments were approved by and performed in compliance with the guidelines of the Institutional Animal Care and Use Committee.

EAE treatment with lipid: 100 μg of PGPC or sphingomyelin or vehicle (0.5% Tween-20 in PBS) was administered in 0.2 ml intravenously in the tail. Treatment with lipid or vehicle was initiated once the PLP₁₃₉₋₁₅₁-immunized mice developed paralysis (representing clinical EAE) and repeated 3, 6, 12, and 18 days later, for a total of five separate injections.

Proliferation and Cytokine Assays.

PLP-stimulated cells from PLP₁₃₉₋₁₅₁-immunized mice administered PGPC prophylactically: SJL/J mice were co-injected with PGPC and PLP₁₃₉₋₁₅₁ in CFA and sacrificed 48 days later. Lymph node cells and splenocytes were then harvested and re-stimulated in vitro (2.5×10⁶ cells/ml) with 10 μg/ml of PLP₁₃₉₋₁₅₁ or with media alone. PLP-stimulated cells from untreated PLP₁₃₉₋₁₅₁-immunized mice: SJL/J mice were immunized with PLP₁₃₉₋₁₅₁ in CFA and sacrificed 10 days later. Splenocytes were then harvested and re-stimulated in vitro (at 5×10⁵ cells/ml) with 10 μg/ml of PLP₁₃₉₋₁₅₁ in the presence of 30 μg/ml of POPS, PGPC, azPC ester, or azPC.

Cd1d^{-/-} T cells: We harvested splenocytes from Cd1d^{-/-} mice and their wild-type littermates and used CD3⁺ T-cell enrichment columns (R&D systems) to isolate T cells. We then stimulated the T cells with plate-bound anti-CD3 antibodies, or plate-bound anti-CD3 plus anti-CD28 antibodies, in the presence of 30 μg/ml of POPS, PGPC, azPC ester, or azPC.

MBP-stimulated cells from MBP_{Ac1-11} transgenic mice: Splenocytes were harvested from mice possessing a transgene encoding a T-cell receptor specific for MBP_{Ac1-11}. 5×10⁶ cells/ml were stimulated in vitro with 2 μg/ml of MBP_{Ac1-11} in the presence of various concentrations of palmitic acid (Sigma), other lipids, or 100% Ethanol (vehicle alone).

Anti-CD3/anti-CD28-stimulated purified T cells: We harvested lymph nodes and spleens from naive C57BL/6 mice and used CD3⁺ T-cell enrichment columns (R&D systems) to isolate CD3⁺ T cells. We then stimulated 1×10⁶ cells/ml of T cells with 5 μg/ml of plate-bound anti-CD3 antibodies plus anti-CD28 antibodies in the presence of 30 μg/ml of POPS, PGPC, azPC ester, brain sulfatides, or azPC, 0.5 mM palmitic acid, or 100% ethanol (as the vehicle control).

LPS-stimulated RAW 264.7 mouse macrophage cells: 1×10⁵ cells/ml of RAW 264.7 cells were stimulated with 100 ng/ml of lipopolysaccharide (LPS) in the presence of 30 μg/ml of POPS, PGPC, azPC ester, or azPC.

Anti-IgM F(ab′)2 fragment/anti-CD40-stimulated purified B cells: We harvested spleens from naive C57BL/6 mice and used a B-cell isolation kit (Miltenyi Biotec) to negatively isolate B cells. We then stimulated 5×10⁵ cells/ml of B cells with 5 μg/ml of each of soluble anti-IgM F(ab′)2 fragment (Jackson ImmunoResearch) antibody and anti-CD40 antibody (eBioscience) in the presence of 30 μg/ml of POPS, PGPC, azPC ester, or azPC.

Phospholipase inhibition: Splenocytes were harvested from mice possessing a transgene encoding a T-cell receptor specific for MBP_{Ac1-11}. 5×10⁶ cells/ml were pre-incubated in complete RPMI media for about 40 min at 37° C. with 5% CO₂, in the presence of the corresponding vehicle (DMSO or 100% ethanol) or a phospholipase inhibitor: OBAA (500 nM, Tocris Bioscience), U 73122 (500 nM, Tocris Bioscience), 525143 (5 μM, EMD, Calbiochem), FKGK 11 (50 μM, Caymen Chemical), YM 26734 (250 μM, Tocris Bioscience), LY 311727 (500 μM, Tocris Bioscience), CAY10590 (200 μM, Caymen Chemical), 525145 (5 μM, EMD, Calbiochem). Afterwards, the splenocytes were plated in triplicate and stimulated in vitro with 2 μg/ml of MBP_{Ac1-11} in the presence of 30 μg/ml azPC for 48 hours. Proliferation response is the ratio of the average triplicate wells of MBP+azPC divided by the average triplicate wells of MBP, or MBP divided by MBP.

All cells (except for RAW 264.7) were cultured in complete RPMI 1640 containing 10% fetal bovine serum supplemented with L-glutamine (2 mM), HEPES (25 mM), sodium pyruvate (1 mM), non-essential amino acids (0.1 mM), penicillin (100 U/ml), streptomycin (0.1 mg/ml), and 2-mercaptoethanol (50 μM). RAW 264.7 cells were cultured in DMEM containing high glucose, 10% fetal bovine serum supplemented with L-glutamine (2 mM), sodium pyruvate (1 mM), and penicillin (100 U/ml), streptomycin (0.1 mg/ml).

For assessment of proliferation, 1 μCi of ³H-thymidine was added to each well for the final 18 hours of culture, and incorporation of radioactivity was quantified using a Betaplate scintillation counter.

Cytokine production by cells from the lymph nodes and spleens of PGPC co-immunized mice was measured after 66 hours of stimulation by using the BD OptEIA™ Mouse IFN-γ ELISA kit (BD Biosciences). Cytokine assays for the anti-CD3/anti-CD28 antibody-stimulated T cells and PLP₁₃₉₋₁₅₁-stimulated splenocytes were performed on culture supernatants after 48 hours of stimulation using the BD OptEIA™ Mouse IL-6, IFN-γ, and TNF alpha ELISA kits (BD Biosciences) and the Mouse IL-17 DuoSet ELISA Development kit (R&D Systems).

Lipidomic Analysis of Brain Samples.

Archived, fresh-frozen, human postmortem samples from MS brain and age-matched healthy brain were analysed by shotgun lipidomics, as previously described. MS brain samples are describe in the main text. Control brain samples were thoroughly examined to rule out the presence of neurological disease. Samples containing MS lesions were dissected and immediately freeze-clamped in liquid nitrogen, pulverized with a stainless-steel mortar and pestle, and their protein concentrations determined by using a BCA protein assay kit (Pierce). Internal standards were added to each tissue sample to enable normalization according to the protein content and quantification relative to that of a selected internal standard through ion intensity comparison (i.e., ratiometric comparison). Each lipid extract was reconstituted in chloroform/methanol (1:1, v/v) at a volume of 500 μL/mg of protein (calculated on the basis of the original protein content of the sample). The lipid extracts were flushed with nitrogen, capped, and stored at −20° C. until used in electrospray ionization mass spectrometric analyses (typically within 1 week). A TSQ Quantum Ultra Plus triple-quadrupole mass spectrometer (Thermo Fisher Scientific) equipped with an automated nanospray apparatus (Nanomate HD, Advion Bioscience Ltd.) and Xcalibur system software were used in the study (45). Each lipid extract solution was diluted to less than 50 pmol of total lipids/μl with CHCl3/MeOH/isopropanol (1:2:4 by volume) before infusion into the mass spectrometer with the nanomate. Typically, a 1 min period of signal averaging was used for each mass spectrum, and a 2 min period of signal averaging for each tandem mass spectrum.

LC-HRMS.

Dried Folch extracts of ˜100 mg brain were resuspended in 500 microliters of 50:50 DCM:MeOH. These were sonicated and centrifuged to remove particles. The authentic standards were prepared at 8-10 ng/ml in 50:50 DCM:MeOH. The chromatography method is based on one described by Oresic. The reversed phase column, which is kept at 50° C., is an Acquity UPLC™ BEH C18 2.1 mm ID×50 mm length with 1.7 μm particles. The binary solvent system includes A. water (1% 1M NH₄Ac, 0.1% HCOOH) and B. acetonitrile/isopropanol (5:2, 1% 1M NH₄Ac, 0.1% HCOOH). The linear gradient starts from 35% B, reaches 100% B in 6 min and remains at this level for the next 7 min. The total run time including a 5 min re-equilibration step is 18 min. The flow rate is 0.200 ml/min and the volume injected 5 μl. The temperature of autosampler is maintained at 4° C. Mass spectra were collected on an Agilent 6530 Q-TOF. MS1 spectra were collected from m/z 400-1000 at a rate of 2 spectra/sec. The gas temperature was 325° C., drying gas was 5 L/min, Nebulizer was 20 psig, capillary voltage was 3500V, nozzle voltage was 2000V, Sheath gas Temp was 325° C. and the Sheath gas flow was 7.5 L/min. For MS/MS spectra, a collision energy of 20V was used with a narrow (1a.m.u.) isolation window; the scan range was m/z 100 to 700 and collected at a rate of 6 spectra/sec.

Flow Cytometry.

Cells were stained according to standard protocols, run on a FACScan flow cytometer (BD Biosciences), and analysed with CellQuest software (BD Immunocytometry Systems) or with FlowJo software version 6.3.2 (Tree Star, Inc.). The antibody conjugates used were FITC anti-CD4 (clone GK1.5, BD Pharmingen), FITC anti-mouse CD8 (clone 53-6.7, BD Pharmingen), FITC anti-mouse CD3 (clone 145-2C11, eBioscience), PE-anti-rat IgG2A isotype control (BD Pharmingen), and PE-anti-mouse CD69 (clone H1.2F3, eBioscience). 7AAD staining was performed by using the Annexin V-PE Apoptosis Detection Kit I (BD Pharmingen).

1. A method for reducing disease severity in a mammalian subject suffering from an autoimmune disease, the method comprising:
administering to said subject a therapeutic dose of a lipid or fatty acid, so as to thereby reduce said disease severity, where the lipid has the structure set forth in Formula I: 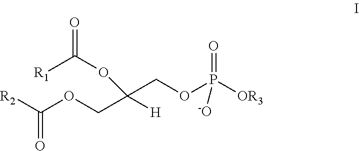
where R₁ and R₂ are independently selected from a linear or branched C₃-C₁₀₀ alkyl; preferably a C₁-C₃₀ alkyl optionally substituted with halo, hydroxy, alkoxy, amino, alkylamino, dialkylamino, sulfate, or phosphate, and which may by saturated, or mono- or di-unsaturated;
R₃ is selected from H, —CH₂CH₂NH₃ (ethan-1-amine), and serine (2-aminobutanoic acid).
R₄ is absent, or when R₃ is H, R₄ may be

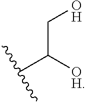 [SMILES: *C(O)CO]

2. The method of claim 1, wherein the lipid has the structure set forth in Formula II:

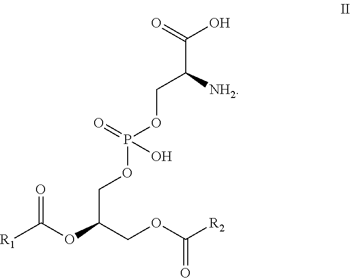

3. The method of claim 1, wherein the lipid has the structure set forth in Formula III:

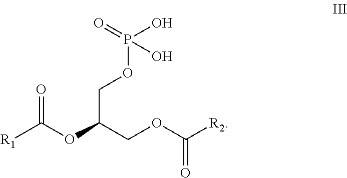

4. The method of claim 1, wherein the lipid has the structure set forth in Formula IV:

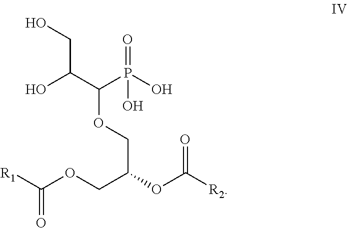

5. The method of claim 1, wherein the lipid has the structure set forth in Formula V:

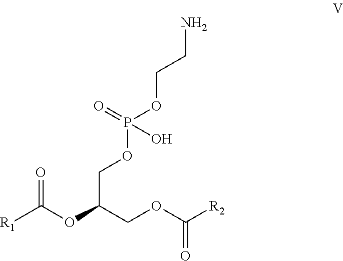

6. The method of claim 1, wherein R₁ or R₂ are (Z)-octadec-9-ene.

7. The method of claim 1, wherein R₁ or R₂ are hexadecane.

8. The method of claim 1, wherein the lipid is selected from (R)-1-(palmitoyloxy)-3-(phosphonooxy)propan-2-yl oleate (POPA); 1-palmitoyl-2-oleoyl-sn-glycero-3-[phospho-l-serine](POPS), 1-palmitoyl-2-oleoyl-sn-glycero-3-phosphoethanolamine (POPE); and 1-palmitoyl-2-oleoyl-sn-glycero-3-phosphoglycerol (POPG).

9. The method of claim 1, wherein the fatty acid is selected from palmitic acid, ethyl palmitate, sebacic acid, octanoic acid, methyl octanoate, and suberic acid.

10. The method of claim 1, wherein said lipid is administered in conjunction with a tolerizing adjuvant.

11. The method of claim 1, wherein said autoimmune disease is a demyelinating disease.

12. The method of claim 11, wherein the demyelinating disease is EAE.

13. The method of claim 11, wherein the demyelinating disease is multiple sclerosis.

14. The method of claim 11, wherein the demyelinating disease is neuromyelitis optica (NMO).

15. The method of claim 11, wherein the demyelinating disease is acute disseminated encephalomyelitis (ADEM).

16. The method of claim 1, wherein the lipid is administered at a dose of from 0.01 mg/kg patient weight to not more than 100 mg/kg.

17. The method according to claim 1, wherein the lipid is administered systemically.

18. The method of according to claim 1, wherein the lipid is administered from 1 to 7 times weekly.

19. The method of claim 16, wherein the lipid is administered every other day.
